(19) United States
(12) Patent Application Publication
(10) Pub. No.: US 2024/0299356 A1
(43) Pub. Date: Sep. 12, 2024

(54) DEVELOPMENT OF PRMT-TARGETING THERAPY TO ENHANCE EGFR-TARGETING DRUG EFFICACY IN NSCLC

(71) Applicant: The Regents of the University of California, Oakland, CA (US)

(72) Inventors: Steven ALTSCHULER, San Francisco, CA (US); Lani WU, San Francisco, CA (US); Xiaoxiao SUN, San Francisco, CA (US); Matthew JACOBSON, San Francisco, CA (US)

(21) Appl. No.: 18/547,871

(22) PCT Filed: Feb. 25, 2022
(86) PCT No.: PCT/US2022/018023

§ 371 Date: Aug. 24, 2023

Related U.S. Application Data
(60) Provisional application No. 63/153,749, filed on Feb. 25, 2021.

(51) Int. Cl. A61K 31/415; A61K 31/40; A61K 31/506; A61K 31/517; A61K 31/555; A61K 31/7068

(52) U.S. Cl. CPC A61K 31/415; A61K 31/40; A61K 31/506; A61K 31/517; A61K 31/555; A61K 31/7068

(57) ABSTRACT

Provided herein are, inter alia, methods and composition for the treatment of cancers that are recalcitrant to treatment and/or become resistant to certain drug treatments. The methods provided may, inter alia, be used to treat cancer in subjects having elevated STAT1 activity levels.

### RELATED APPLICATION DATA

This application claims the benefit of priority under 35 U.S.C. § 119(e) of the U.S. Patent Application No. 63/153,749, filed on Feb. 25, 2021, which is hereby incorporated by reference in its entirety and for all purposes.

### BACKGROUND

Cancer remains an important set of therapeutic targets. Some cancer types are more recalcitrant to therapy. Additionally, incomplete killing of cancer cells is a major challenge for the treatment of cancers. Small populations of cancer cells, not killed by an initial treatment, can multiply created a recurrence of the cancer, and create a population of cancer cells that are no longer susceptible to treatment with the chosen line of treatment. Provided herein are methods and compositions that address these and other needs in the art.

### BRIEF SUMMARY

In an aspect is provided a method of treating cancer in a subject having elevated STAT1 activity, the method including administering a therapeutically effective amount of a type I PRMT inhibitor to the subject.

In an aspect is provided a method of treating cancer in a subject, the method including administering a therapeutically effective amount of a type I PRMT inhibitor to the subject, wherein the subject has been previously treated with a STAT1 activating compound.

In an aspect is provided a method of treating cancer in a subject, the method including administering a therapeutically effective amount of a type I PRMT inhibitor and a STAT1 activating compound to the subject.

In an aspect is provided a method of treating cancer in a subject in need thereof, the method including: (i) detecting an elevated STAT1 activity in a subject; and (ii) administering a therapeutically effective amount of a type I PRMT inhibitor to the subject.

In an aspect is provided a method of treating cancer in a subject in need thereof, the method including: (i) detecting an elevated STAT1 activity in a subject; and (ii) administering a therapeutically effective amount of an anti-cancer agent to the subject.

In an aspect is provided a method of treating cancer in a subject in need thereof, the method including: (i) detecting a STAT1 activity in a subject; and (ii) administering a therapeutically effective amount of a STAT1 activating compound to the subject.

### BRIEF DESCRIPTION OF THE DRAWINGS

FIGS. 1A-1F present exemplary data showing that Type I PRMT inhibition reduced IFNγ signaling-modulated drug tolerance. FIG. 1A present a three-step approach aimed at investigating drug tolerance. FIG. 1B is a line graph showing PC9 cell viabilities under six-day treatment of various doses of erlotinib in the context of IFNγ. FIGS. 1C and 1D are line graphs showing that the inhibition of type I PRMTs by small molecule inhibitor MS023 (FIG. 1C) and PRMT1-targeting siRNA (FIG. 1D) reduced drug tolerance in the PC9/erlotinib model system. FIG. 1E is a line graph showing that inhibition of type I PRMTs by MS023 reduced cancer drug tolerance specifically in the IFNγ/STAT1 activation context (normalized to DMSO controls in corresponding IFNγ contexts). Tolerance model for FIG. 1E: PC9 treated with 2.5 μM erlotinib for six days. FIG. 1F shows that the inhibition of type I PRMTs by MS023 prevented the emergence of cancer drug-tolerant persisters specifically in the IFNγ/STAT1 activation context. Tolerance model: PC9 treated with 2.5 μM erlotinib.

FIGS. 2A-2H present exemplary data showing that Type I PRMT inhibition reduced drug tolerance via restraining STAT1 protein synthesis. FIG. 2A is a line graph showing that knockout of STAT1 by CRISPR blocked IFNγ-induced change of drug-tolerant subpopulation size. FIG. 2B is a bar graph showing that JAK inhibitor ruxolitinib dose-dependently increased drug tolerance in the context of IFNγ. FIG. 2C is a bar graph showing that show that heterozygous deletion of STAT1 (+/−) by CRISPR in PC9 decreased tolerance (tolerance model: PC9 treated with 2.5 μM erlotinib). FIG. 2D is a line graph showing homozygous knockout of STAT1 by CRISPR (tolerance model: PC9 treated with 2.5 μM erlotinib). FIG. 2E shows that the inhibition of type I PRMTs by MS023 reduced the expression of STAT1 protein. FIG. 2F shows that show that the knockdown of poly(A)-binding protein 2 (PABP2), reduced STAT1 protein expression in PC9. FIG. 2G shows that type I PRMTs methylated PABP2 at multiple arginine residues. Relative intensities were measured from by mass spectrometry experiments. FIG. 2H is a diagram of STAT1-mediated drug tolerance and type I PRMT vulnerability to eliminate drug tolerance.

FIGS. 3A-3D present exemplary data showing that Type I PRMT inhibition enhanced cancer drug efficacy. FIG. 3A presents line graphs showing that type I PRMT inhibitor MS023 reduced drug tolerance in multiple cancer models, including erlotinib-treated lung cancers (top panel), osimertinib-treated lung cancers (middle panel), carboplatin-treated lung cancers (bottom panel). FIGS. 3B-3D present data showing the correlation between endogenous STAT1 expression levels and the efficacy of type I PRMT inhibition. FIG. 3B shows the baseline STAT1 expression of lung cancer cells from CCLE database. Gray dots indicate cell lines that were tested for the efficacies of type I PRMT inhibition (by MS023). FIG. 3C shows dose-response curves of MS023 on various lung cancer drug tolerance models. FIG. 3D shows that MS023 efficacy (measured by area under the curve (AUC) in FIG. 3C) correlated with endogenous STAT1 expression levels of the cells.

FIGS. 4A-4F present exemplary data showing that Type I PRMT inhibition reduces IFNγ signaling-modulated drug tolerance. FIG. 4A is a line graph showing the pro-survival effects of IFNγ in a variety of cell types with the cytotoxic inducers. Six-day tolerance assay in lung cancer cells was performed under 2.5 μM erlotinib or 50 μM carboplatin. FIG. 4B is a line graph showing PC9 cells co-treated with IFNγ and cytotoxic stressors (EGFR inhibitor Gefitinib, PARP inhibitor olaparib, death ligand TRAIL, procaspase-3 activator PAC-1), and cell viabilities were measured by Cell TiterGlo after six days. FIG. 4C is a bar-graph showing PRMT1 knockdown efficiency measured by qPCR after two days of siRNA transfection. FIG. 4D is a line graph showing that inhibition of type I PRMTs by MS023 reduced cancer drug tolerance specifically in the IFNγ/STAT1 activation context (normalized to DMSO control in (−) IFNγ context). Tolerance model for FIG. 4D: PC9 treated with 2.5 μM erlotinib for six days. FIG. 4E is a line graph showing that inhibition of type I PRMTs by GSK3368715 reduced cancer drug tolerance specifically in the IFNγ/STAT1 activation context (normalized to DMSO controls in corresponding IFNγ contexts). FIG. 4F is a line graph line graphs showing that inhibition of type I PRMTs by GSK3368715 reduced cancer drug tolerance specifically in the IFNγ/STAT1 activation context (normalized to DMSO control in (−) IFNγ context). Tolerance model for FIGS. 4E and 4F: PC9 treated with 2.5 μM erlotinib for six days.

FIGS. 5A-5R present exemplary data showing that Type I PRMT inhibition reduced drug tolerance via restraining STAT1 protein synthesis. FIG. 5A shows that reactivation of major EGFR bypass signaling (AKT, ERK and STAT3 pathway) by IFNγ was not observed. FIG. 5B shows a Western blot of the time course of STAT1 signaling activation stimulated by IFNγ in PC9 cells. FIG. 5C is a line graph showing that STAT1 modulated cancer drug tolerance, and knockdown of STAT1 by siRNA blocked IFNγ-induced change of drug-tolerant subpopulation size. FIG. 5D shows the offset of STAT1 and P-STAT1 activations, exemplified in PC9 cells. FIG. 5E is a bar graph showing that cancer drug treatment (EGFR drugs and other cytotoxic stressors) enhanced STAT1 expression across multiple cell lines. FIG. 5F shows that JAK inhibitor ruxolitinib dose-dependently decreased P-STAT1 expression. FIG. 5G shows that heterozygous deletion of STAT1 (+/−) by CRISPR in PC9 decreased STAT1 expression. Tolerance model for FIG. 5G: PC9 treated with 2.5 μM erlotinib. FIG. 5H presents exemplary data showing differential gene expression in erlotinib-tolerant PC9 cells comparing to parental cells. The top panel of FIG. 5H is a volcano plot in which IRDS genes are shown in black. The bottom panel of FIG. 5H is a line graph showing the accumulative distribution of adjusted p-values of IRDS genes (gray) versus all genes excluding IRDS genes (black). FIG. 5I is a line graph showing the knockdown of STAT1 by siRNA. FIG. 5J is a line graph showing the homozygous knockout of STAT1 by CRISPR. Tolerance model for FIGS. 5I and 5J: PC9 treated with 2.5 μM erlotinib. FIG. 5K shows that inhibition of type I PRMTs by MS023 or GSK3368715 downregulated total STAT1 protein level; this was observed independent of whether IFNγ or erlotinib was present. FIG. 5L shows that the inhibition of type I PRMTs by GSK3368715 reduced the expression of STAT1 protein. FIG. 5M is a bar graph showing that the inhibition of type I PRMTs by MS023 did not reduce STAT1 mRNA. FIG. 5N shows that the inhibition of type I PRMTs reduced STAT1 protein synthesis. PC9 cells were pretreated with MS023 for one day, followed by co-treatment of erlotinib, and perturbations of protein synthesis or degradation for 16 hours. Cycloheximide (CHX): protein synthesis inhibitor. Bortezomib (Bort): proteasome inhibitor. Ammonium chloride (NH₄Cl)): lysosomal function inhibitor. FIG. 5O shows that the knockdown of PABP2 reduced the expression of STAT1, but not of other signaling proteins that were tested. FIG. 5P is a bar graph showing type I PRMTs methylate PABP2 at multiple arginine residues. Relative intensities were measured from by mass spectrometry experiments. FIG. 5Q shows spectra of identified peptides of PABP2 containing mono-methylated (first panel from top) and dimethylated (second panel from top) R17 by mass spectrometry, and spectra of identified peptides of PABP2 containing mono-methylated (third panel from top) and dimethylated (fourth panel from top) R238 by mass spectrometry. FIG. 5R shows that knockdown of PABP2 reduced the expression of STAT1, but not of other signaling proteins that were tested.

FIGS. 6A-6E present exemplary data showing that Type I PRMT inhibition enhances cancer drug efficacy. FIG. 6A is a line graphs showing that type I PRMT inhibitor MS023 reduced drug tolerance in gemcitabine-treated pancreatic cancers. FIG. 6B shows that PRMT1 was found to be ubiquitously abundant across different cell lines (compared to STAT1 expression). FIG. 6C is a plot showing that the inhibition of type I PRMTs reduced drug tolerance in MTAP wild type cells as well as MTAP deficient cells. FIG. 6D shows that PRMT1 was found to be ubiquitously abundant across different cell lines (compared to STAT1 expression). FIG. 6E shows differential efficacies of type I PRMT inhibition.

FIG. 7 shows a list of cell line models tested and their MTAP statuses

FIG. 8 is a Western blot of MTAP evaluation of various cancer cell lines.

FIG. 9 shows that the inhibition of type I PRMTs by MS023 reduced the proliferation of solid tumors with wild type splicing factors (e.g. SRSF2, SF3B1, U2AF1).

FIG. 10 shows a table of the IC₅₀ values of MS023 on reducing drug tolerance in multiple cancer models.

FIG. 11 shows that the tolerance-reducing efficacies of MS023 (measured by area under the curve (AUC)) did not correlate with MDR1 expressions. Each dot indicates a cancer cell line model.

FIGS. 12A-12B show that type I PRMT inhibition by MS023 or GSK3368715 reduced the expression of immune checkpoint protein PD-L1 in lung cancer cell MGH119 (FIG. 12A) and pancreatic cancer cell MIAPACA2 (FIG. 12B).

### DETAILED DESCRIPTION

### Definitions

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. The disease may be an autoimmune disease. The disease may be an inflammatory disease. The disease may be an infectious disease. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatinifori carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the interal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid) used to treat cancer through destruction or inhibition of cancer cells or tissues. Anticancer agents may be selective for certain cancers or certain tissues. In embodiments, anticancer agents herein may include epigenetic inhibitors and multi-kinase inhibitors.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Gudrin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Selective” or “selectivity” or the like of a compound refers to the compound's ability to discriminate between molecular targets (e.g. a compound having selectivity toward HMT SUV39H1 and/or HMT G9a).

“Specific”, “specifically”, “specificity”, or the like of a compound refers to the compound's ability to cause a particular action, such as inhibition, to a particular molecular target with minimal or no action to other proteins in the cell (e.g. a compound having specificity towards HMT SUV39H1 and/or HMT G9a displays inhibition of the activity of those HMTs whereas the same compound displays little-to-no inhibition of other HMTs such as DOT1, EZH1, EZH2, GLP, MLL1, MLL2, MLL3, MLL4, NSD2, SET1b, SET7/9, SET8, SETMAR, SMYD2, SUV39H2).

The terms “immune response” and the like refer, in the usual and customary sense, to a response by an organism that protects against disease. The response can be mounted by the innate immune system or by the adaptive immune system, as well known in the art.

The terms “modulating immune response” and the like refer to a change in the immune response of a subject as a consequence of administration of an agent, e.g., a compound as disclosed herein, including embodiments thereof. Accordingly, an immune response can be activated or deactivated as a consequence of administration of an agent, e.g., a compound as disclosed herein, including embodiments thereof.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “memory T cell” is a T cell that has previously encountered and responded to its cognate antigen during prior infection, encounter with cancer or previous vaccination. At a second encounter with its cognate antigen memory T cells can reproduce (divide) to mount a faster and stronger immune response than the first time the immune system responded to the pathogen.

A “regulatory T cell” or “suppressor T cell” is a lymphocyte which modulates the immune system, maintains tolerance to self-antigens, and prevents autoimmune disease.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the compound provided herein when used separately from the therapeutic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the therapeutic agent when used separately from the compound provided herein.

The term “immune response” used herein encompasses, but is not limited to, an “adaptive immune response”, also known as an “acquired immune response” in which adaptive immunity elicits immunological memory after an initial response to a specific pathogen or a specific type of cells that is targeted by the immune response, and leads to an enhanced response to that target on subsequent encounters. The induction of immunological memory can provide the basis of vaccination.

The term “immunogenic” or “antigenic” refers to a compound or composition that induces an immune response, e.g., cytotoxic T lymphocyte (CTL) response, a B cell response (for example, production of antibodies that specifically bind the epitope), an NK cell response or any combinations thereof, when administered to an immunocompetent subject. Thus, an immunogenic or antigenic composition is a composition capable of eliciting an immune response in an immunocompetent subject. For example, an immunogenic or antigenic composition can include one or more immunogenic epitopes associated with a pathogen or a specific type of cells that is targeted by the immune response. In addition, an immunogenic composition can include isolated nucleic acid constructs (such as DNA or RNA) that encode one or more immunogenic epitopes of the antigenic polypeptide that can be used to express the epitope(s) (and thus be used to elicit an immune response against this polypeptide or a related polypeptide associated with the targeted pathogen or type of cells).

An “inhibitor” refers to a compound (e.g. compounds described herein) that reduces activity when compared to a control, such as absence of the compound or a compound with known inactivity.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-activator interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” “activating compound,” etc. refer to a substance capable of detectably increasing the expression or activity of a given gene or protein. The agonist can increase expression or activity by 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “inhibitor,” “repressor” or “antagonist” or “downregulator” interchangeably refer to a substance capable of detectably decreasing the expression or activity of a given gene or protein. The antagonist can decrease expression or activity by 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

A “PD-1 protein” or “PD-1” as referred to herein includes any of the recombinant or naturally-occurring forms of the Programmed cell death protein 1 (PD-1) also known as cluster of differentiation 279 (CD 279) or variants or homologs thereof that maintain PD-1 protein activity (e.g. within at least 50%, 80%, 90%, 95%, 96%, 97%, 98%, 99% or 100% activity compared to PD-1 protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-1 protein. In embodiments, the PD-1 protein is substantially identical to the protein identified by the UniProt reference number Q15116 or a variant or homolog having substantial identity thereto. In embodiments, the PD-1 protein is substantially identical to the protein identified by the UniProt reference number Q02242 or a variant or homolog having substantial identity thereto.

A “PD-L1” or “PD-L1 protein” as referred to herein includes any of the recombinant or naturally-occurring forms of programmed death ligand 1 (PD-L1) also known as cluster of differentiation 274 (CD 274) or variants or homologs thereof that maintain PD-L1 activity (e.g. within at least 50%, 80%, 90%, 95%, 96%, 97%, 98%, 99% or 100% activity compared to PD-L1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PD-L1 protein. In embodiments, the PD-L1 protein is substantially identical to the protein identified by the UniProt reference number Q9NZQ7 or a variant or homolog having substantial identity thereto.

The term “CD4” as referred to herein is a glycoprotein expressed on the surface of T helper cells, regulatory T cells, monocytes, macrophages, and dendritic cells. CD4 was originally known as leu-3 and T4 (after the OKT4 monoclonal antibody). CD4 as referred to herein has four immunoglobulin domains (D₁ to D₄) that are exposed on the extracellular surface of the cell, see ENTREZ No. 920, UNIPROT No. P01730, and GENBANK® Accession No. NP-000607, which are incorporated by reference.

The term “CD8” as referred to herein is a transmembrane glycoprotein that serves as a co-receptor for the T cell receptor (TCR). Like the TCR, CD8 binds to a major histoconpatibility complex (MHIC) molecule, but is specific for the class I MHC protein, see ENTREZ No. 925 and UNIPROT No. P01732, which are incorporated by reference herein.

The terms “TAP” and “transporter associated with antigen processing” as used herein refer to a protein complex which is part of the ATP-binding-cassette transporter family. TAP is a heterodimer formed by two proteins: TAP-1 (encoded by the TAP1 gene) and TAP-2 (encoded by the TAP2 gene).TAP transports cytosolic peptides into the endoplasmic reticulum, where they bind MHC class 1 molecules and are subsequently presented on the surface of the cell for CD8⁺ T-cell recognition. Thus, TAP plays an important role in the regulation of CD8⁺ T-cell recognition and aberrant TAP activity has been observed in cancer. In embodiments, the TAP-1 protein is substantially identical to the protein identified by the UniProt reference number Q03518 or a variant or homolog having substantial identity thereto. In embodiments, the TAP-2 protein is substantially identical to the protein identified by the UniProt reference number Q03419 or a variant or homolog having substantial identity thereto.

The terms “CIITA” and “class II, major histocompability complex, transactivator” as used herein refer to a protein encoded by the C2TA gene. mRNA expression of CIITA is only detected in human leukocyte antigen (HLA) system class II-positive cells and tissues. CIITA is a transcriptional coactivator and functions through the activation of the RFX5 transcription factor. In the nucleus, CIITA acts as a positive regulator of class II major histocompatibility complex gene transcripton. CIITA expression is induced by IFNγ and is upregulated under inflammatory conditions. In embodiments, the CIITA protein is substantially identical to the protein identified by the UniProt reference number P33076 or a variant or homolog having substantial identity thereto.

The terms “HLA-DR” and “human leukocyte antigen—DR isotype” as used herein refer to a major histocompatiability complex (MHC) class II cell surface receptor encoded by the human leukocyte antigen complex on chromosome 6. A complex of HLA-DR and peptide act as a ligand for the T-cell receptor. HLA-DR has a role in the presentation of peptide antigens to the immune system to either stimulate or suppress T-cell responses. HLA-DR is upregulated with immune system activity and, thus, is a marker for immune stimulation. Aberrant HLA-DR activity is observed in inflammatory and autoimmune disease.

The terms “HLA-DMA” and “human leukocyte antigen—DM alpha chain” as used herein refer to a protein encoded by the HLA-DMA gene. HLA-DMA is a member of the HLA class II alpha chain paralogues. HLA-DMA is the alpha chain component of the intracellular HLA-DM protein which plays a role in the antigen presentation mechanism in antigen presenting cells of the immune system. Impairment in HL-DM function can lead to inflammatory and autoimmune disease. In embodiments, the HLA-DMA protein is substantially identical to the protein identified by the UniProt reference number P28067 or a variant or homolog having substantial identity thereto.

The term “PRMT” as referred to herein is a protein arginine N-methyltransferase. PRMTs are enzymes that catalyze the methylation of arginine residues within proteins, resulting in changes in several biological processes such as RNA regulation, signal transduction, and chromatin regulation. As histones are common substrates of PRMTs, their methylation can alter the histone code. The modified structure of histones results in changes in gene expression by altering their interaction with other proteins and by generating docking sites for chromatin-associated proteins. Dysregulation of this group of enzymes can result in aberrant gene expression which may eventually lead to human disease. The activity of PRMTs has been implicated in stem cell pluripotency, cancer metastasis, and tumorigenesis. In humans, nine PRMTs have been identified so far with the type I arginine methyltransferases consisting of PRMT1, PRMT2, PRMT3, PRMT4, PRMT6, and PRMT8, type II consisting of PRMT5, and PRMT9 that catalyze the formation of asymmetric dimethylarginine derivative (ADMA) and symmetric dimethylarginine derivative (SDMA), respectively. The type III enzyme consists of PRMT7 that only catalyzes the formation of monomethylated Arginine (MMA). An example of a PRMT5 inhibitor is GSK3326595 from GSK (Epizyme) which is in phase 1 clinicial trials.

The terms “IRF1” and “Interferon regulatory factor 1” as used herein refer to a protein that is encoded by the IRF1 gene. The protein encoded by this gene is a transcriptional regulator and tumor suppressor, serving as an activator of genes involved in both innate and acquired immune responses. The encoded protein activates the transcription of genes involved in the body's response to viruses and bacteria, playing a role in cell proliferation, apoptosis, the immune response, and DNA damage response. This protein represses the transcription of several other genes. As a tumor suppressor, it both suppresses tumor cell growth and stimulates an immune response against tumor cells. Defects in this gene have been associated with gastric cancer, myelogenous leukemia, and lung cancer. In embodiments, the IRF1 protein is substantially identical to the protein identified by the UniProt reference number P10914 or a variant or homolog having substantial identity thereto.

The terms “SOCS1” and “Suppressor of Cytokine Signaling” as used herein refer to a protein that is encoded by the SOCS1 gene. This gene encodes a member of the STAT-induced STAT inhibitor (SSI), also known as suppressor of cytokine signaling (SOCS), family. SSI family members are cytokine-inducible negative regulators of cytokine signaling. The expression of this gene can be induced by a subset of cytokines, including IL2, IL3 erythropoietin (EPO), CSF2/GM-CSF, and interferon (IFN)-gamma. The protein encoded by this gene functions downstream of cytokine receptors, and takes part in a negative feedback loop to attenuate cytokine signaling. Knockout studies in mice suggested the role of this gene as a modulator of IFN-gamma action, which is required for normal postnatal growth and survival. In embodiments, the SOCS1 protein is substantially identical to the protein identified by the UniProt reference number 015524 or a variant or homolog having substantial identity thereto.

The terms “PIAS” and “protein inhibitor of activated STAT” as used herein refer to a member of the PIAS protein family. There are four members of the PIAS family. PIAS proteins act as transcriptional co-regulators with numerous substrates, including STAT proteins. PIAS proteins have E3 SUMO-protein ligase activity. PIAS proteins act as inhibitors of STAT1 signaling.

The terms “TCPTP”, “T cell tyrosine phosphatase”, “protein tyrosine phosphatase non-receptor 2”, and “PTPN2” as used herein refer to a protein encoded by the PTPN2 gene. This gene encodes a protein phosphatase that targets JAK, STAT, EGFR and other receptor tyrosine kinases. TCPTP dephosphorylizes its substrates, thereby reducing their signaling. TCPTP is a regulator of glucose metabolism, inflammation, cancer processes. In embodiments, the PTPN2 protein is substantially identical to the protein identified by the UniProt reference number P17706 or a variant or homolog having substantial identity thereto.

The terms “APOL1”, “APOL-1” and “Apolipoprotein L1” as used herein refer to the protein encoded by the APOL1 gene. This gene encodes a secreted high density lipoprotein which binds to apolipoprotein A-I. Apolipoprotein A-I is a relatively abundant plasma protein and is the major apoprotein of HDL. It is involved in the formation of most cholesteryl esters in plasma and also promotes efflux of cholesterol from cells. This apolipoprotein L family member may play a role in lipid exchange and transport throughout the body, as well as in reverse cholesterol transport from peripheral cells to the liver. Several different transcript variants encoding different isoforms have been found for this gene. In embodiments, the APOL1 protein is substantially identical to the protein identified by the UniProt reference number 014791 or a variant or homolog having substantial identity thereto.

The terms “B2M” and “Beta-2-Microglobulin” as used herein refer to a protein encoded by the B2M gene. This gene encodes a serum protein found in association with the major histocompatibility complex (MHC) class I heavy chain on the surface of nearly all nucleated cells. The protein has a predominantly beta-pleated sheet structure that can form amyloid fibrils in some pathological conditions. The encoded antimicrobial protein displays antibacterial activity in amniotic fluid. A mutation in this gene has been shown to result in hypercatabolic hypoproteinemia. In embodiments, the B2M protein is substantially identical to the protein identified by the UniProt reference number P61769 or a variant or homolog having substantial identity thereto.

The terms “GBP” and “Guanylate-Binding Protein” as used herein refer to a protein that is a member of the guanylate-binding protein family. The guanylate-binding protein family is a family of GTPases that is induced by interferon (IFN)-gamma. GTPases induced by IFN-gamma (Interferon-inducible GTPase) are key to the protective immunity against microbial and viral pathogens. These GTPases are classified into three groups: the small 47-KD immunity-related GTPases (IRGs), the Mx proteins (MX1, MX2), and the large 65- to 67-KD GTPases. Guanylate-binding proteins (GBP) fall into the last class. In humans, there are seven GBPs (hGBP1-7). Structurally, hGBP1 consists of two domains: a compact globular N-terminal domain harboring the GTPase function, and an alpha-helical finger-like C-terminal domain. Human GBP1 is secreted from cells without the need of a leader peptide, and has been shown to exhibit antiviral activity against Vesicular stomatitis virus and Encephalomyocarditis virus, as well as being able to regulate the inhibition of proliferation and invasion of endothelial cells in response to IFN-gamma. In embodiments, the GBP1 protein is substantially identical to the protein identified by the UniProt reference number P32455 or a variant or homolog having substantial identity thereto.

The terms “RNF213” and “Ring Finger Protein 213” as used herein refer to a protein encoded by the RNF213 gene. This gene encodes a protein containing a C3HC4-type RING finger domain, which is a specialized type of Zn-finger that binds two atoms of zinc and is thought to be involved in mediating protein-protein interactions. The protein also contains an AAA domain, which is associated with ATPase activity. This gene is a susceptibility gene for Moyamoya disease, a vascular disorder of intracranial arteries. This gene is also a translocation partner in anaplastic large cell lymphoma and inflammatory myofibroblastic tumor cases, where a t(2;17)(p23;q25) translocation has been identified with the anaplastic lymphoma kinase (ALK) gene on chromosome 2, and a t(8;17)(q24;q25) translocation has been identified with the MYC gene on chromosome 8. Alternative splicing results in multiple transcript variants. In embodiments, the RNF213 protein is substantially identical to the protein identified by the UniProt reference number Q63HN8 or a variant or homolog having substantial identity thereto.

The terms “interferon alpha”, “IFNα” and “IFN-α” as used herein refer to a dimerized soluble cytokine that is a member of the type I class of interferons. There are 12 functional human IFNα proteins which exhibit high homology in primary, secondary, and tertiary structures. IFNα is a cytokine that is critical for innate and adaptive immunity against viral, some bacterial and protozoal infections. IFNα is an important modulator for the function of B cells, T effector cells, and regulatory T cells. Aberrant IFNα expression is associated with a number of autoinflammatory and autoimmune diseases. IFNα is an important stimulator of antiviral genes which prevent viral replication within target cells and a key regulator of the innate immune response. IFNα can influence dendritic cell activation, maturation, migration, and survival. In addition IFNα can directly enhance natural killer (NK) cells, T cells, and B cells activity. IFNα is produced predominantly plasmacytoid dendritic cells, but IFNα is secreted by natural killer (NK), B cells, T cells, and macrophages. In embodiments, the IFNα protein is substantially identical to the protein identified by the UniProt reference number P01562 or a variant or homolog having substantial identity thereto.

The terms “interferon gamma”, “type II interferon”, “IFNγ” and “IFN-γ” as used herein refer to a dimerized soluble cytokine that is the only member of the type II class of interferons. IFNγ is a cytokine that is critical for innate and adaptive immunity against viral, some bacterial and protozoal infections. IFNγ is an important activator of macrophages and inducer of Class II major histocompatibility complex (MHC) molecule expression. Aberrant IFNγ expression is associated with a number of autoinflammatory and autoimmune diseases. The importance of IFNγ in the immune system stems in part from its ability to inhibit viral replication directly, and most importantly from its immunostimulatory and immunomodulatory effects. IFNγ is produced predominantly by natural killer (NK) and natural killer T (NKT) cells as part of the innate immune response, and by CD4 Th1 and CD8 cytotoxic T lymphocyte (CTL) effector T cells once antigen-specific immunity develops as part of the adaptive immune response. IFNγ is also produced by non-cytotoxic innate lymphoid cells (ILC). In embodiments, the IFNγ protein is substantially identical to the protein identified by the UniProt reference number P01579 or a variant or homolog having substantial identity thereto.

The terms “epidermal growth factor” and “EGF” as used herein refer to a protein encoded by the EGF gene. This gene encodes a secreted protein which is a member of the EGF family of growth factors. EGF is an important regulator of cell division, proliferation, and survival. EGF binds the epidermal growth factor receptor (EGFR) on the surface of cells. In embodiments, the EGF protein is substantially identical to the protein identified by the UniProt reference number P01133 or a variant or homolog having substantial identity thereto.

The terms “EGFR protein” and “EGFR” as used herein includes any of the recombinant or naturally-occurring forms of epidermal growth factor receptor (EGFR) also known as ErbB-1 or HER1 in humans, or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR protein. In embodiments, the EGFR protein is substantially identical to the protein identified by the UniProt reference number P00533 or a variant or homolog having substantial identity thereto.

The terms “platelet-derived growth factor” and “PDGF” as used herein refer to a protein encoded by the PDGF gene. This gene encodes a secreted protein which is a member of the PDGF family of growth factors. PDGF is a regulator of cell growth and division and plays a key role in the formation of blood vessels. PDGF is predominantly synthesized, stored and secreted by platelets, but other cells can also produce PDGF. PDGF binds the platelet-derived growth factor receptor (PDGFR) on the surface of cells. PDGF is a dimeric glycoprotein that can be composed of two PDGF-A subunits, two PDGF-B subunits, or a combination thereof. In embodiments, the PDGF-A subunit of a PDGF protein is substantially identical to the protein identified by the UniProt reference number P04085 or a variant or homolog having substantial identity thereto. In embodiments, the PDGF-B subunit of a PDGF protein is substantially identical to the protein identified by the UniProt reference number P01127 or a variant or homolog having substantial identity thereto.

The terms “interleukin 6,” “IL-6,” and “IL6” as used herein refer to a protein encoded by the IL6 gene. IL6, a cytokine protein, is secreted by macrophages in response to pathogen-associated molecular patterns, thus activating the innate immune response. IL6 is responsible for stimulating acute protein synthesis and neutrophil production. In addition, IL6 supports B cell growth and inhibits regulatory T cells. IL6 can interact with several proteins and/or receptors including interleukin-6 receptor, glycoprotein 130, and galectin-3. Aberrant IL6 signaling plays a role in inflammatory and autoimmune disease. In embodiments, the IL6 protein is substantially identical to the protein identified by the UniProt reference number P05231 or a variant or homolog having substantial identity thereto.

The terms “STAT1” and “Signal transducer and activator of transcription 1” refer to is a transcription factor which in humans is encoded by the STAT1 gene. The protein encoded by this gene is a member of the STAT protein family. In response to cytokines and growth factors, STAT family members are phosphorylated by the receptor associated kinases, and then form homo- or heterodimers that translocate to the cell nucleus where they act as transcription activators. This protein can be activated by various ligands including interferon-alpha, interferon-gamma, EGF, PDGF and IL6. This protein mediates the expression of a variety of genes, which is thought to be important for cell viability in response to different cell stimuli and pathogens. Two alternatively spliced transcript variants encoding distinct isoforms have been described.

The terms “genetic mutation” or “mutation” as used herein refers to is an alteration in the nucleotide sequence of the genome of an organism, virus, or extrachromosomal DNA. Mutations result from errors during DNA or viral replication, mitosis, or meiosis or other types of damage to DNA (such as pyrimidine dimers caused by exposure to ultraviolet radiation), which then may undergo error-prone repair (especially microhomology-mediated end joining), cause an error during other forms of repair, or cause an error during replication (translesion synthesis). Mutations may also result from insertion or deletion of segments of DNA due to mobile genetic elements. Mutation can result in many different types of change in sequences. Mutations in genes can have no effect, alter the product of a gene, or prevent the gene from functioning properly or completely. Mutations can also occur in nongenic regions. Mutations can be large scale mutations in chromosomal structure which include: amplifications (or gene duplications) or repetition of a chromosomal segment or presence of extra piece of a chromosome broken piece of a chromosome which may become attached to a homologous or non-homologous chromosome; deletions of large chromosomal regions, leading to loss of the genes within those regions; mutations whose effect is to juxtapose previously separate pieces of DNA, potentially bringing together separate genes to form functionally distinct fusion genes; large scale changes to the structure of chromosomes called chromosomal rearrangement that can lead to a decrease of fitness but also to speciation in isolated, inbred populations (e.g. chromosomal translocations, chromosomal inversions, reversing the orientation of a chromosomal segment, non-homologous chromosomal crossover, or interstitial deletion), and the loss of heterozygosity (the loss of one allele, either by a deletion or a genetic recombination event, in an organism that previously had two different alleles). For instance, a chromosomal rearrangement can be an “inactivating rearrangement” if it affects a gene which becomes inactivated, when the protein it encodes has less or no function (being partially or wholly inactivated) when compared to the original (not mutated) gene product. Small scale mutations affect a gene in one or a few nucleotides and include: insertions (addition of one or more extra nucleotides into the DNA), deletions (removal of one or more nucleotides from the DNA) and substitution mutations (exchange of one or more nucleotides for one or more other nucleotides).

The terms “JAK” and “Janus Kinase” as used herein refer to a family of intracellular, non-receptor tyrosine kinases that transduce cytokine-mediated signals via the JAK-STAT pathway. The JAKs possess two near-identical phosphate-transferring domains: one domain exhibits the kinase activity, while the other negatively regulates the kinase activity of the first. The four JAK family members are: Janus kinase 1 (JAK1), Janus kinase 2 (JAK2), Janus kinase 3 (JAK3) and Tyrosine kinase 2 (TYK2).

The terms “IFNGR” and “Interferon-Gamma Receptor” as used herein refer to a protein complex is the heterodimer of two chains: Interferon gamma receptor 1 (encoded by the IFNGR1 gene, also known as CD119 (Cluster of Differentiation 119)) and Interferon gamma receptor 2 (encoded by the IFNGR2 gene, also known as IFN-γR2). IFNGR1 encodes IFN-γR1, which is the ligand-binding chain (alpha) of the heterodimeric gamma interferon receptor, which is found on macrophages. IFNGR2, encodes IFN-γR2, the non-ligand-binding beta chain of the gamma interferon receptor. Human interferon-gamma receptor is a multimer of two IFN-γR1 chains (encoded by IFNGR1) and two IFN-γR2 chains.

The term “9p21.33 disruption” as used herein refer to mutations in the NTRK2 gene. Tropomyosin receptor kinase B (TrkB), also known as tyrosine receptor kinase B, or BDNF/NT-3 growth factors receptor or neurotrophic tyrosine kinase, receptor, type 2 is a protein that in humans is encoded by the NTRK2 gene. TrkB is a receptor for brain-derived neurotrophic factor (BDNF). This gene was identified as playing a role in human cancers because of the identification of NTRK2 (TrkB) gene fusions and other oncogenic alterations in a number of tumor types.

The terms “CDKN2A deletion” as used herein refer to deletion mutations in the CDKN2A gene. CDKN2A, also known as cyclin-dependent kinase inhibitor 2A, is a gene which in humans is located at chromosome 9, band p21.3. It is ubiquitously expressed in many tissues and cell types. The gene codes for two proteins, including the INK4 family member p16 (or p16INK4a) and p14arf. Both act as tumor suppressors by regulating the cell cycle. p16 inhibits cyclin dependent kinases 4 and 6 (CDK4 and CDK6) and thereby activates the retinoblastoma (Rb) family of proteins, which block traversal from G1 to S-phase. p14ARF (known as p19ARF in the mouse) activates the p53 tumor suppressor. Somatic mutations of CDKN2A are common in the majority of human cancers, with estimates that CDKN2A is the second most commonly inactivated gene in cancer after p53. Germline mutations of CDKN2A are associated with familial melanoma, glioblastoma and pancreatic cancer. The CDKN2A gene also contains one of 27 SNPs associated with increased risk of coronary artery disease.

The terms “5q deletion” as used herein refer to the loss of part of the long arm (q arm, band 5q33.1) of human chromosome 5. The human chromosome 5 is responsible for many forms of growth and development (cell divisions). Alterations to this chromosome changes may cause cancers. Th 5q deletion chromosome abnormality is most commonly associated with the myelodysplastic syndrome, a hematological disorder observed in bone marrow myelocyte cells.

### Method of Treatment

Provided herein are, inter alia, methods and composition for the treatment of cancers (e.g., lung cancer) that are recalcitrant to treatment and/or become resistant to certain drug treatments. The methods provided may, inter alia, be used to treat cancer in subjects having elevated STAT1 activity levels. In embodiments, the cancer is lung cancer.

A “STAT1 activity” as provided herein refers to a level of STAT1 expression, STAT1 phosphorylation, and/or expression of one or more STAT1 signaling pathway proteins, including, for example, IFN, IFN receptor, JAK, IRF, SOCS, PIAS, TCPTP. In embodiments, the STAT1 activity is a level of STAT1 expression, STAT1 phosphorylation, a level of expression of one or more STAT1 signaling pathway proteins, for example, IFN, IFN receptor, JAK, IRF, SOCS, PIAS, TCPTP, and any combinations thereof. A STAT1 activity may also include a ratio of levels, such as a ratio of phosphorylated STAT1 to unphosphorylated STAT1, and/or a ratio between two STAT1 signaling pathway proteins. In embodiments, the cancer cell for treatment has an activity of one or more STAT1 signaling pathway proteins such as IFN, IFN receptor, JAK, IRF, SOCS, PIAS, TCPTP, and any combinations thereof. In embodiments, a level of infiltration of immune cells into the tumor or tumor microenvironment is indicative of a STAT1 activity.

In embodiments, the STAT1 activity is a higher score, a higher percentage of positive stain cells, or a higher fold level expression in the tumor sample relative to a standard control. A standard control may be a matched cell from a human, a matched tissue from a human, a cell of the same origin as the tumor but known to have low or no detectable STAT1 activation, or the same tumor sample (such as stained with nonimmune serum control). In embodiments, a STAT1 activity is a level of about 2, 2.5, 3, or more than 3 fold higher than a standard control. Methods for measuring a STAT1 level include methods for measuring RNA expression and protein expression. These methods include, but are not limited to, sequencing, reverse transcription polymerase chain reaction (RT-PCR), fluorescence-based in situ hybridization (FISH), histological staining, immunohistochemistry (IHC), Western blot analysis, liquid chromatography-mass spectrometry (LC-MS).

An “elevated STAT1 activity” as provided herein refers to an increased level of STAT1 function relative to a standard control. In embodiments, the elevated STAT1 activity is an increased concentration or increased level of the protein relative to the concentration or level of a standard control. In embodiments, the elevated STAT1 activity is an increased stimulation of the STAT1 signaling pathway relative to a standard control. In embodiments, the elevated STAT1 activity is an increased signal transduction of the STAT1 signaling pathway relative to a standard control. In embodiments, the elevated STAT1 activity is an increased enzymatic activity of a STAT1 signaling pathway component relative to a standard control. For the embodiments provided herein a “STAT1 signaling pathway component” includes, without limitation, any STAT1 protein, homolog or variant thereof, any downstream protein targeted by STAT1 (i.e., whose biological activity is modulated by STAT1) and any upstream protein that modulates STAT1 activity or expression in a detectable way using standard methods well known in the chemical and biological arts.

In embodiments, the elevated STAT1 activity is an increased gene expression of a STAT1 signaling pathway component relative to a standard control. In embodiments, the elevated STAT1 activity is an increased transcription of a STAT1 signaling pathway component relative to a standard control. In embodiments, the elevated STAT1 activity is an increased gene translation of a STAT1 signaling pathway component relative to a standard control. In embodiments, the elevated STAT1 activity of a STAT1 signaling pathway component is increased by 5%, 10%, 20%, 30%, 40%, 50%, 60%, 70%, 80%, 90% or more in comparison to a standard control. In embodiments, the elevated STAT1 activity of a STAT1 signaling pathway component is about 5%, 10%, 20%, 30%, 40%, 50%, 60%, 70%, 80%, 90% or more in comparison to a standard control. In embodiments, the elevated STAT1 activity of a STAT1 signaling pathway component is 100%, 200%, 300%, 400%, 500%, 600%, 700%, 800%, or 900% more in comparison to a standard control. In embodiments, the elevated STAT1 activity of a STAT1 signaling pathway component is about 100%, 200%, 300%, 400%, 500%, 600%, 700%, 800%, or 900% more in comparison to a standard control.

In embodiments, the elevated STAT1 activity of a STAT1 signaling pathway component is 5% more in comparison to a standard control. In embodiments, the elevated STAT1 activity of a STAT1 signaling pathway component is 10% more in comparison to a standard control. In embodiments, the elevated STAT1 activity of a STAT1 signaling pathway component is 20% more in comparison to a standard control. In embodiments, the elevated STAT1 activity of a STAT1 signaling pathway component is 30% more in comparison to a standard control. In embodiments, the elevated STAT1 activity of a STAT1 signaling pathway component is 40% more in comparison to a standard control. In embodiments, the elevated STAT1 activity of a STAT1 signaling pathway component is 50% more in comparison to a standard control. In embodiments, the elevated STAT1 activity of a STAT1 signaling pathway component is 60% more in comparison to a standard control. In embodiments, the elevated STAT1 activity of a STAT1 signaling pathway component is 70% more in comparison to a standard control. In embodiments, the elevated STAT1 activity of a STAT1 signaling pathway component is 80% more in comparison to a standard control. In embodiments, the elevated STAT1 activity of a STAT1 signaling pathway component is 90% more in comparison to a standard control.

In embodiments, the elevated STAT1 activity of a STAT1 signaling pathway component is about 5% more in comparison to a standard control. In embodiments, the elevated STAT1 activity of a STAT1 signaling pathway component is about 10% more in comparison to a standard control. In embodiments, the elevated STAT1 activity of a STAT1 signaling pathway component is about 20% more in comparison to a standard control. In embodiments, the elevated STAT1 activity of a STAT1 signaling pathway component is about 30% more in comparison to a standard control. In embodiments, the elevated STAT1 activity of a STAT1 signaling pathway component is about 40% more in comparison to a standard control. In embodiments, the elevated STAT1 activity of a STAT1 signaling pathway component is about 50% more in comparison to a standard control. In embodiments, the elevated STAT1 activity of a STAT1 signaling pathway component is about 60% more in comparison to a standard control. In embodiments, the elevated STAT1 activity of a STAT1 signaling pathway component is about 70% more in comparison to a standard control. In embodiments, the elevated STAT1 activity of a STAT1 signaling pathway component is about 80% more in comparison to a standard control. In embodiments, the elevated STAT1 activity of a STAT1 signaling pathway component is about 90% more in comparison to a standard control.

In embodiments, the elevated STAT1 activity of a STAT1 signaling pathway component is 100% more in comparison to a standard control. In embodiments, the elevated STAT1 activity of a STAT1 signaling pathway component is 200% more in comparison to a standard control. In embodiments, the elevated STAT1 activity of a STAT1 signaling pathway component is 300% more in comparison to a standard control. In embodiments, the elevated STAT1 activity of a STAT1 signaling pathway component is 400% more in comparison to a standard control. In embodiments, the elevated STAT1 activity of a STAT1 signaling pathway component is 500% more in comparison to a standard control. In embodiments, the elevated STAT1 activity of a STAT1 signaling pathway component is 600% more in comparison to a standard control. In embodiments, the elevated STAT1 activity of a STAT1 signaling pathway component is 700% more in comparison to a standard control. In embodiments, the elevated STAT1 activity of a STAT1 signaling pathway component is 800% more in comparison to a standard control. In embodiments, the elevated STAT1 activity of a STAT1 signaling pathway component is 900% more in comparison to a standard control.

In embodiments, the elevated STAT1 activity of a STAT1 signaling pathway component is about 100% more in comparison to a standard control. In embodiments, the elevated STAT1 activity of a STAT1 signaling pathway component is about 200% more in comparison to a standard control. In embodiments, the elevated STAT1 activity of a STAT1 signaling pathway component is about 300% more in comparison to a standard control. In embodiments, the elevated STAT1 activity of a STAT1 signaling pathway component is about 400% more in comparison to a standard control. In embodiments, the elevated STAT1 activity of a STAT1 signaling pathway component is about 500% more in comparison to a standard control. In embodiments, the elevated STAT1 activity of a STAT1 signaling pathway component is about 600% more in comparison to a standard control. In embodiments, the elevated STAT1 activity of a STAT1 signaling pathway component is about 700% more in comparison to a standard control. In embodiments, the elevated STAT1 activity of a STAT1 signaling pathway component is about 800% more in comparison to a standard control. In embodiments, the elevated STAT1 activity of a STAT1 signaling pathway component is about 900% more in comparison to a standard control.

In embodiments, the elevated STAT1 activity of a STAT1 signaling pathway component is 1.5-fold, 2-fold, 3-fold, 4-fold, 5-fold, 10-fold or 100-fold higher relative to a standard control. In embodiments, the elevated STAT1 activity of a STAT1 signaling pathway component is about 1.5-fold, 2-fold, 3-fold, 4-fold, 5-fold, 10-fold or 100-fold higher relative to a standard control. In embodiments, the elevated STAT1 activity of a STAT1 signaling pathway component is 150-fold, 200-fold, 300-fold, 400-fold, 500-fold, 1000-fold or 10,000-fold higher relative to a standard control. In embodiments, the elevated STAT1 activity of a STAT1 signaling pathway component is about 150-fold, 200-fold, 300-fold, 400-fold, 500-fold, 1000-fold or 10,000-fold higher relative to a standard control.

In embodiments, the elevated STAT1 activity of a STAT1 signaling pathway component is 1.5-fold higher relative to a standard control. In embodiments, the elevated STAT1 activity of a STAT1 signaling pathway component is 2-fold higher relative to a standard control. In embodiments, the elevated STAT1 activity of a STAT1 signaling pathway component is 3-fold higher relative to a standard control. In embodiments, the elevated STAT1 activity of a STAT1 signaling pathway component is 4-fold higher relative to a standard control. In embodiments, the elevated STAT1 activity of a STAT1 signaling pathway component is 5-fold higher relative to a standard control. In embodiments, the elevated STAT1 activity of a STAT1 signaling pathway component is 10-fold higher relative to a standard control. In embodiments, the elevated STAT1 activity of a STAT1 signaling pathway component is 25-fold higher relative to a standard control. In embodiments, the elevated STAT1 activity of a STAT1 signaling pathway component is 50-fold higher relative to a standard control. In embodiments, the elevated STAT1 activity of a STAT1 signaling pathway component is 75-fold higher relative to a standard control. In embodiments, the elevated STAT1 activity of a STAT1 signaling pathway component is 100-fold higher relative to a standard control. In embodiments, the elevated STAT1 activity of a STAT1 signaling pathway component is 200-fold higher relative to a standard control. In embodiments, the elevated STAT1 activity of a STAT1 signaling pathway component is 300-fold higher relative to a standard control. In embodiments, the elevated STAT1 activity of a STAT1 signaling pathway component is 400-fold higher relative to a standard control. In embodiments, the elevated STAT1 activity of a STAT1 signaling pathway component is 500-fold higher relative to a standard control. In embodiments, the elevated STAT1 activity of a STAT1 signaling pathway component is 1000-fold higher relative to a standard control. In embodiments, the elevated STAT1 activity of a STAT1 signaling pathway component is 10,000-fold higher relative to a standard control.

In embodiments, the elevated STAT1 activity of a STAT1 signaling pathway component is about 1.5-fold higher relative to a standard control. In embodiments, the elevated STAT1 activity of a STAT1 signaling pathway component is about 2-fold higher relative to a standard control. In embodiments, the elevated STAT1 activity of a STAT1 signaling pathway component is about 3-fold higher relative to a standard control. In embodiments, the elevated STAT1 activity of a STAT1 signaling pathway component is about 4-fold higher relative to a standard control. In embodiments, the elevated STAT1 activity of a STAT1 signaling pathway component is about 5-fold higher relative to a standard control. In embodiments, the elevated STAT1 activity of a STAT1 signaling pathway component is about 10-fold higher relative to a standard control. In embodiments, the elevated STAT1 activity of a STAT1 signaling pathway component is about 25-fold higher relative to a standard control. In embodiments, the elevated STAT1 activity of a STAT1 signaling pathway component is about 50-fold higher relative to a standard control. In embodiments, the elevated STAT1 activity of a STAT1 signaling pathway component is about 75-fold higher relative to a standard control. In embodiments, the elevated STAT1 activity of a STAT1 signaling pathway component is about 100-fold higher relative to a standard control. In embodiments, the elevated STAT1 activity of a STAT1 signaling pathway component is about 200-fold higher relative to a standard control. In embodiments, the elevated STAT1 activity of a STAT1 signaling pathway component is about 300-fold higher relative to a standard control. In embodiments, the elevated STAT1 activity of a STAT1 signaling pathway component is about 400-fold higher relative to a standard control. In embodiments, the elevated STAT1 activity of a STAT1 signaling pathway component is about 500-fold higher relative to a standard control. In embodiments, the elevated STAT1 activity of a STAT1 signaling pathway component is about 1000-fold higher relative to a standard control. In embodiments, the elevated STAT1 activity of a STAT1 signaling pathway component is about 10,000-fold higher relative to a standard control.

In embodiments, the STAT1 signaling pathway component is a downstream gene induced by STAT1 activation (e.g., IRF1, SOCS1, APOL1, B2M, GBPs, RNF213). In embodiments, the STAT1 signaling pathway component is a downstream gene induced by STAT1 activation (e.g. IRF1, SOCS1, APOL1, B2M, GBPs, RNF213) in combination with STAT1 or interferon-gamma (e.g., IFN, IFN receptor, JAK, IRF, SOCS, PIAS, TCPTP).

In embodiments, the elevated STAT1 activity is an increased STAT1 gene expression relative to a standard control. In embodiments, the elevated STAT1 activity is an increased STAT1 gene transcription relative to a standard control. In embodiments, the elevated STAT1 activity is an increased STAT1 gene translation relative to a standard control. In embodiments, the elevated STAT1 activity is increased by 10%, 20%, 30%, 40%, 50%, 60%, 70%, 80%, 90% or more in comparison to a standard control. In embodiments, the elevated STAT1 activity is 1.5-fold, 2-fold, 3-fold, 4-fold, 5-fold, 10-fold or 100-fold higher relative to standard control.

In embodiments, the elevated STAT1 activity is a genetic variation of a STAT1 signaling pathway component (e.g., JAK1/2 mutation, IFNGR1/2 mutation, B2M mutation, copy number loss of the interferon gene cluster due to 9p21.33 disruption, CDKN2A deletion, 5q deletion, IRF1 inactivating rearrangement and deletion, and SOCS1 mutation or amplification). A genetic variation as provided herein includes point mutations, gene truncations, deletions, amplifications, or gene fusions; the variations could be somatic mutations or germline mutations and could be heterozygous or homozygous. Where the elevated STAT1 activity is a genetic variation of a STAT1 signaling pathway component, the presence of the genetic variation of a STAT1 signaling pathway component causes the elevated STAT1 activity.

In embodiments, the elevated STAT1 activity is the infiltration of immune cells into the tumor or tumor microenvironment (for example the detection of CD4⁺ T cells, CD8⁺ T cells, macrophages, neutrophils, or NK cells in tumor biopsy samples), a detectable expression level of immune co-stimulatory molecules (for example the detection of PD-1, PD-L1/2, CD155, CD80/86, CD28, CTLA-4, galectin-9, TIM3, Siglec-15, ICOS, ICOS-L, CD47, CD70, or 4-1BBL expression in tumor biopsy samples), or a detectable expression level of antigen presentation molecules (for example the detection of TAP, B2M, CIITA, HLA-DR, or HLA-DMA expression in tumor biopsy samples), as measured by bulk sample score, positive cell count, or greater expression (e.g. 2-fold higher) relative to a standard control. Where the elevated STAT1 activity is the infiltration of immune cells into the tumor or tumor microenvironment, the infiltration of immune cells into the tumor or tumor microenvironment is indicative of elevated STAT1 activity.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.). The “expression level,” “amount,” or “level,” or used herein interchangeably, of STAT1, IFNα, IFNγ, EGF, PDGF, or IL6 is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

Expression levels can be measured by methods known to one skilled in the art and also disclosed herein. Expression levels can be measured, for example, by measuring RNA expression and protein expression. These methods include, but are not limited to, sequencing, reverse transcription polymerase chain reaction (RT-PCR), Western blot analysis, fluorescence-based in situ hybridization (FISH), histological staining, immunohistochemistry (IHC), liquid chromatography-mass spectrometry (LC-MS) using assays and techniques suitable for measuring RNA levels. For example, a RNA-Seq kit can be used to measure expression levels and is suitable for kits as described herein. Exemplary technologies useful in measuring expression levels herein include, but are not limited to, RNA ACCESS® protocol or TRUSEQ® RIBO-ZERO® protocol (ILLUMINA®)), RT-qPCR, qPCR, multiplex qPCR (e.g. fluidigm), nanostring technologies, RT-qPCR, microarray analysis, SAGE, or MassARRAY.

A “standard control” as provided herein refers to a sample that serves as a reference, usually a known reference, for comparison to a test sample. For example, a test sample can be taken from a patient suspected of having a disease (e.g., cancer) and compared to samples from a patient known to have the disease, or a known normal (non-disease) individual. A control can also represent an average value gathered from a population of similar individuals, e.g., disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

In embodiments, a standard control is a level of STAT1 expression from a sample or subject lacking the disease, a sample or subject at a selected stage of the disease or disease state, or in the absence of a particular variable such as a therapeutic agent. Alternatively, the control includes a known amount STAT1 expression. Such a known amount correlates with an average level of subjects lacking the disease, at a selected stage of the disease or disease state, or in the absence of a particular variable such as a therapeutic agent. A control also includes the level of STAT1 expression from one or more selected samples or subjects as described herein. For example, a control includes the level of STAT1 expression in a sample from a subject that does not have the disease, is at a selected stage of disease or disease state, or has not received treatment for the disease. Another exemplary control level includes an assessment of the level of STAT1 expression in samples taken from multiple subjects that do not have the disease, are at a selected stage of the disease, or have not received treatment for the disease.

When the control level of STAT1 expression includes the level of STAT1 expression in a sample or subject in the absence of a therapeutic agent, the control sample or subject is optionally the same sample or subject to be tested before or after treatment with a therapeutic agent or is a selected sample or subject in the absence of the therapeutic agent. Alternatively, a standard control is an average expression level calculated from a number of subjects without a particular disease. A control level also includes a known control level or value known in the art.

A “standard control” as used herein in reference to the expression level of one or more genes (e.g., STAT1, IRF1, SOCS1, APOL1, B2M, GBP, RNF213) refers to the expression level measured in a control subject (e.g. in a sample from the control subject) or population of control subjects. In embodiments, the control subject is a healthy subject relative to the subject being tested, wherein the healthy subject does not have cancer. In embodiments, the control subject is a healthy subject relative to the subject being tested, wherein the healthy subject does not have breast cancer. In embodiments, the control subject is a test subject prior to treatment of the test subject, wherein the test subject and control subject have cancer. For example, in embodiments, the test subject has been treated for cancer with an anticancer agent and the control subject is the test subject prior to treatment. In embodiments, the population of control subjects is a diverse collection of healthy subjects and diseased subjects, wherein the expression level of the test subject is compared to the expression levels of the population of control subjects (e.g. an average of expression levels of the population of control subjects). In embodiments, the population of control subjects is a collection of healthy subjects that do not have cancer, wherein the expression level of the test subject is compared to the expression levels of the population of control subjects (e.g. an average of expression levels of the population of control subjects). In embodiments, the population of control subjects is a collection of subjects that have been treated for cancer, wherein the expression level of the test subject is compared to the expression levels of the population of control subjects (e.g. an average of expression levels of the population of control subjects). In embodiments, the control subject and the test subject are the same. In further embodiments, the standard control is the STAT1 expression level in a sample from healthy tissue. In embodiments, the healthy tissue is adjacent to the cancer tissue. In further embodiments, the standard control is that STAT1 expression level in a blood sample.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue extracts such as homogenized tissue, tumor tissue, cellular extracts, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, and combinations thereof.

By “tissue sample” or “cell sample” is meant a collection of similar cells obtained from a tissue of a subject or individual. The source of the tissue or cell sample may be solid tissue as from a FFPE, FF, fresh, frozen, and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease (e.g., cancer) tissue/organ. The tissue sample may contain compounds which are not naturally intermixed with the tissue in nature such as preservatives, anticoagulants, buffers, fixatives, nutrients, antibiotics, or the like.

In an aspect is provided a method of treating cancer in a subject having elevated STAT1 activity, the method including administering a therapeutically effective amount of a type I PRMT inhibitor to the subject.

A “STAT1 gene” as referred to herein includes any of the recombinant or naturally-occurring forms of the gene encoding Signal transducer and Activator of transcription 1 (STAT1) or variants thereof that maintain STAT1 expression (e.g. within at least 50%, 80%, 90%, 95%, 96%, 97%, 98%, 99% or 100% expression level compared to STAT1). In some aspects, variants have at least 90%, 95%, 96%, 97%, 98%, 99% or 100% nucleotide sequence identity across the whole sequence or a portion of the sequence (e.g. a 50, 100, 150 or 200 continuous nucleotide portion) compared to a naturally occurring STAT1 gene. In embodiments, the STAT1 gene is substantially identical to the nucleic acid identified by the NCBI reference number Gene ID: 6772 or a variant having substantial identity thereto.

The term “STAT1” or “STAT1 protein” as provided herein includes any of the recombinant or naturally-occurring forms of the Signal transducer and Activator of transcription 1 (STAT1) or variants or homologs thereof that maintain STAT1 protein activity (e.g. within at least 50%, 80%, 90%, 95%, 96%, 97%, 98%, 99% or 100% activity compared to STAT1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring STAT1 polypeptide. In embodiments, STAT1 is the protein as identified by the UniProtKB/Swiss-Prot sequence reference P42224.2, homolog or functional fragment thereof.

A “PRMT1 gene” or “PRMT gene” as referred to herein includes any of the recombinant or naturally-occurring forms of the gene encoding protein arginine methyltransferase 1 (PRMT1) or variants thereof that maintain PRMT1 expression (e.g. within at least 50%, 80%, 90%, 95%, 96%, 97%, 98%, 99% or 100% expression level compared to PRMT1). In some aspects, variants have at least 90%, 95%, 96%, 97%, 98%, 99% or 100% nucleotide sequence identity across the whole sequence or a portion of the sequence (e.g. a 50, 100, 150 or 200 continuous nucleotide portion) compared to a naturally occurring PRMT1 gene. In embodiments, the PRMT1 gene is substantially identical to the nucleic acid identified by the NCBI reference number Gene ID: 3276 or a variant having substantial identity thereto.

The term “PRMT1” or“PRMT1 protein” as provided herein includes any of the recombinant or naturally-occurring forms of the protein arginine methyltransferase 1 (PRMT1) or variants or homologs thereof that maintain PRMT1 protein activity (e.g. within at least 50%, 80%, 90%, 95%, 96%, 97%, 98%, 99% or 100% activity compared to PRMT1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PRMT1 polypeptide. In embodiments, PRMT1 is the protein as identified by the UniProtKB/Swiss-Prot sequence reference Q99873, homolog or functional fragment thereof.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The terms “type 1 PRMT inhibitor,” or “PRMT1 inhibitor” interchangeably refer to a substance capable of detectably decreasing the expression or activity of a PRMT1 gene or a PRMT1 protein. The PRMT1 inhibitor decreases PRMT1 expression or PRMT1 activity by 5%, 10%, 20%, 30%, 40%, 50%, 60%, 70%, 80%, 90% or more in comparison to a standard control in the absence of the inhibitor. In embodiments, the PRMT1 inhibitor decreases PRMT1 expression or PRMT1 activity by 5% in comparison to a standard control (e.g., the absence of a PRMT1 inhibitor). In embodiments, the PRMT1 inhibitor decreases PRMT1 expression or PRMT1 activity by 10% in comparison to a standard control (e.g., the absence of a PRMT1 inhibitor). In embodiments, the PRMT1 inhibitor decreases PRMT1 expression or PRMT1 activity by 20% in comparison to a standard control (e.g., the absence of a PRMT1 inhibitor). In embodiments, the PRMT1 inhibitor decreases PRMT1 expression or PRMT1 activity by 30% in comparison to a standard control (e.g., the absence of a PRMT1 inhibitor). In embodiments, the PRMT1 inhibitor decreases PRMT1 expression or PRMT1 activity by 40% in comparison to a standard control (e.g., the absence of a PRMT1 inhibitor). In embodiments, the PRMT1 inhibitor decreases PRMT1 expression or PRMT1 activity by 50% in comparison to a standard control (e.g., the absence of a PRMT1 inhibitor). In embodiments, the PRMT1 inhibitor decreases PRMT1 expression or PRMT1 activity by 60% in comparison to a standard control (e.g., the absence of a PRMT1 inhibitor). In embodiments, the PRMT1 inhibitor decreases PRMT1 expression or PRMT1 activity by 70% in comparison to a standard control (e.g., the absence of a PRMT1 inhibitor). In embodiments, the PRMT1 inhibitor decreases PRMT1 expression or PRMT1 activity by 80% in comparison to a standard control (e.g., the absence of a PRMT1 inhibitor). In embodiments, the PRMT1 inhibitor decreases PRMT1 expression or PRMT1 activity by 90% in comparison to a standard control (e.g., the absence of a PRMT1 inhibitor).

In embodiments, the PRMT1 inhibitor decreases PRMT1 expression or PRMT1 activity by about 5%, 10%, 20%, 30%, 40%, 50%, 60%, 70%, 80%, 90% or more in comparison to a standard control. In embodiments, the PRMT1 inhibitor decreases PRMT1 expression or PRMT1 activity by about 5% in comparison to a standard control (e.g., the absence of a PRMT1 inhibitor). In embodiments, the PRMT1 inhibitor decreases PRMT1 expression or PRMT1 activity by about 10% in comparison to a standard control (e.g., the absence of a PRMT1 inhibitor). In embodiments, the PRMT1 inhibitor decreases PRMT1 expression or PRMT1 activity by about 20% in comparison to a standard control (e.g., the absence of a PRMT1 inhibitor). In embodiments, the PRMT1 inhibitor decreases PRMT1 expression or PRMT1 activity by about 30% in comparison to a standard control (e.g., the absence of a PRMT1 inhibitor). In embodiments, the PRMT1 inhibitor decreases PRMT1 expression or PRMT1 activity by about 40% in comparison to a standard control (e.g., the absence of a PRMT1 inhibitor). In embodiments, the PRMT1 inhibitor decreases PRMT1 expression or PRMT1 activity by about 50% in comparison to a standard control (e.g., the absence of a PRMT1 inhibitor). In embodiments, the PRMT1 inhibitor decreases PRMT1 expression or PRMT1 activity by about 60% in comparison to a standard control (e.g., the absence of a PRMT1 inhibitor). In embodiments, the PRMT1 inhibitor decreases PRMT1 expression or PRMT1 activity by about 70% in comparison to a standard control (e.g., the absence of a PRMT1 inhibitor). In embodiments, the PRMT1 inhibitor decreases PRMT1 expression or PRMT1 activity by about 80% in comparison to a standard control (e.g., the absence of a PRMT1 inhibitor). In embodiments, the PRMT1 inhibitor decreases PRMT1 expression or PRMT1 activity by about 90% in comparison to a standard control (e.g., the absence of a PRMT1 inhibitor).

In embodiments, PRMT1 expression or PRMT1 activity is 1.5-fold, 2-fold, 3-fold, 4-fold, 5-fold, 10-fold or 100-fold lower than the PRMT1 expression or PRMT1 activity in the absence of the PRMT1 inhibitor. In embodiments, PRMT1 expression or PRMT1 activity is 1.5-fold lower relative to a standard control (e.g., the absence of a PRMT1 inhibitor). In embodiments, PRMT1 expression or PRMT1 activity is 2-fold lower relative to a standard control (e.g., the absence of a PRMT1 inhibitor). In embodiments, PRMT1 expression or PRMT1 activity is 3-fold lower relative to a standard control (e.g., the absence of a PRMT1 inhibitor). In embodiments, PRMT1 expression or PRMT1 activity is 4-fold lower relative to a standard control (e.g., the absence of a PRMT1 inhibitor). In embodiments, PRMT1 expression or PRMT1 activity is 5-fold lower relative to a standard control (e.g., the absence of a PRMT1 inhibitor). In embodiments, PRMT1 expression or PRMT1 activity is 10-fold lower relative to a standard control (e.g., the absence of a PRMT1 inhibitor). In embodiments, PRMT1 expression or PRMT1 activity is 100-fold lower relative to a standard control (e.g., the absence of a PRMT1 inhibitor).

In embodiments, PRMT1 expression or PRMT1 activity is about 1.5-fold, 2-fold, 3-fold, 4-fold, 5-fold, 10-fold or 100-fold lower than the PRMT1 expression or PRMT1 activity in the absence of the PRMT1 inhibitor. In embodiments, PRMT1 expression or PRMT1 activity is about 1.5-fold lower relative to a standard control (e.g., the absence of a PRMT1 inhibitor). In embodiments, PRMT1 expression or PRMT1 activity is about 2-fold lower relative to a standard control (e.g., the absence of a PRMT1 inhibitor). In embodiments, PRMT1 expression or PRMT1 activity is about 3-fold lower relative to a standard control (e.g., the absence of a PRMT1 inhibitor). In embodiments, PRMT1 expression or PRMT1 activity is about 4-fold lower relative to a standard control (e.g., the absence of a PRMT1 inhibitor). In embodiments, PRMT1 expression or PRMT1 activity is about 5-fold lower relative to a standard control (e.g., the absence of a PRMT1 inhibitor). In embodiments, PRMT1 expression or PRMT1 activity is about 10-fold lower relative to a standard control (e.g., the absence of a PRMT1 inhibitor). In embodiments, PRMT1 expression or PRMT1 activity is about 100-fold lower relative to a standard control (e.g., the absence of a PRMT1 inhibitor).

A type I protein arginine methyltransferase inhibitor (also referred to herein as PRMT type I inhibitor, type I protein arginine methyltransferase inhibitor and PRMTi) refers to an agent that inhibits any one or more of the following: protein arginine methyltransferase 1 (PRMT1), protein arginine methyltransferase 2 (PRMT2), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments of the methods herein, a PRMT1 inhibits PRMT1, the major isoform of type I protein arginine methyltransferase.

The term MS023 as used herein refers to the human type I protein arginine methyltransferase (PRMT) inhibitor having the structure of formula:

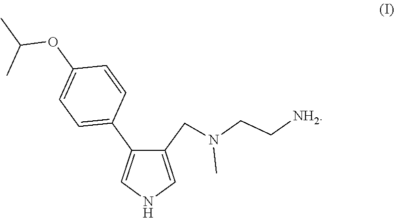 [SMILES: CC(C)Oc1ccc(-c2c[nH]cc2CN(C)CCN)cc1]

In a customary sense, MS023 refers to the compound identified by CAS Reg. No. 1831110-54-3.

The term GSK3368715 as used herein refers to the human type I PRMT inhibitor having the structure of formula:

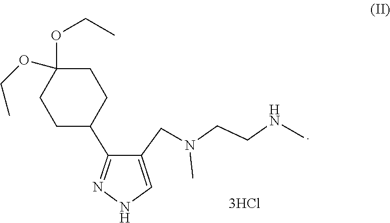 [SMILES: CCOC1(OCC)CCC(c2n[nH]cc2CN(C)CCNC)CC1]

In a customary sense, GSK3368715 as used herein refers to the compound identified by CAS Reg. No. 2227587-25-7.

In embodiments, the subject is or has been treated with an anti-cancer agent. In embodiments, the anti-cancer agent is erlotinib, osimertinib, carboplatin and/or gemcitabine. In embodiments, the anti-cancer agent is erlotinib. In embodiments, the anti-cancer agent is osimertinib. In embodiments, the anti-cancer agent is carboplatin. In embodiments, the anti-cancer agent is gemcitabine. In embodiments, the anti-cancer agent is erlotinib, osimertinib, carboplatin and gemcitabine. In embodiments, the anti-cancer agent is erlotinib, osimertinib, carboplatin or gemcitabine. In embodiments, the anti-cancer agent is an anti-cancer agent selected from erlotinib, osimertinib, carboplatin, and gemcitabine.

In embodiments, the anti-cancer agent is administered at a therapeutically effective amount well known in the art to treat cancer. In embodiments, the anti-cancer agent is administered at a concentration of 1 μM to 50 μM. In embodiments, the anti-cancer agent is administered at a concentration of 2 μM to 50 μM. In embodiments, the anti-cancer agent is administered at a concentration of 5 μM to 50 μM. In embodiments, the anti-cancer agent is administered at a concentration of 10 μM to 50 μM. In embodiments, the anti-cancer agent is administered at a concentration of 15 μM to 50 μM. In embodiments, the anti-cancer agent is administered at a concentration of 20 μM to 50 μM. In embodiments, the anti-cancer agent is administered at a concentration of 25 μM to 50 μM. In embodiments, the anti-cancer agent is administered at a concentration of 30 μM to 50 μM. In embodiments, the anti-cancer agent is administered at a concentration of 35 μM to 50 μM. In embodiments, the anti-cancer agent is administered at a concentration of 40 μM to 50 μM. In embodiments, the anti-cancer agent is administered at a concentration of 45 μM to 50 μM.

In embodiments, the anti-cancer agent is administered at a concentration of 1 μM to 45 μM. In embodiments, the anti-cancer agent is administered at a concentration of 1 μM to 40 μM. In embodiments, the anti-cancer agent is administered at a concentration of 1 μM to 35 μM. In embodiments, the anti-cancer agent is administered at a concentration of 1 μM to 30 μM. In embodiments, the anti-cancer agent is administered at a concentration of 1 μM to 25 μM. In embodiments, the anti-cancer agent is administered at a concentration of 1 μM to 20 μM. In embodiments, the anti-cancer agent is administered at a concentration of 1 μM to 15 μM. In embodiments, the anti-cancer agent is administered at a concentration of 1 μM to 10 μM. In embodiments, the anti-cancer agent is administered at a concentration of 1 μM to 5 μM. In embodiments, the anti-cancer agent is administered at a concentration of 1 μM to 2 μM. In embodiments, the anti-cancer agent is administered at a concentration of 1 μM, 2 μM, 5 μM, 10 μM, 15 μM, 20 μM, 25 μM, 30 μM, 35 μM, 40 μM, 45 μM or 50 μM.

In embodiments, the anti-cancer agent is administered at a concentration of about 1 μM to about 50 μM. In embodiments, the anti-cancer agent is administered at a concentration of about 2 μM to about 50 μM. In embodiments, the anti-cancer agent is administered at a concentration of about 5 μM to about 50 μM. In embodiments, the anti-cancer agent is administered at a concentration of about 10 μM to about 50 μM. In embodiments, the anti-cancer agent is administered at a concentration of about 15 μM to about 50 μM. In embodiments, the anti-cancer agent is administered at a concentration of about 20 μM to about 50 μM. In embodiments, the anti-cancer agent is administered at a concentration of about 25 μM to about 50 μM. In embodiments, the anti-cancer agent is administered at a concentration of about 30 μM to about 50 μM. In embodiments, the anti-cancer agent is administered at a concentration of about 35 μM to about 50 μM. In embodiments, the anti-cancer agent is administered at a concentration of about 40 μM to about 50 μM. In embodiments, the anti-cancer agent is administered at a concentration of about 45 μM to about 50 μM.

In embodiments, the anti-cancer agent is administered at a concentration of about 1 μM to about 45 μM. In embodiments, the anti-cancer agent is administered at a concentration of about 1 μM to about 40 μM. In embodiments, the anti-cancer agent is administered at a concentration of about 1 μM to about 35 μM. In embodiments, the anti-cancer agent is administered at a concentration of about 1 μM to about 30 μM. In embodiments, the anti-cancer agent is administered at a concentration of about 1 μM to about 25 μM. In embodiments, the anti-cancer agent is administered at a concentration of about 1 μM to about 20 μM. In embodiments, the anti-cancer agent is administered at a concentration of about about 1 μM to about 15 μM. In embodiments, the anti-cancer agent is administered at a concentration of about 1 μM to about 10 μM. In embodiments, the anti-cancer agent is administered at a concentration of about 1 μM to about 5 μM. In embodiments, the anti-cancer agent is administered at a concentration of about 1 μM to about 2 μM. In embodiments, the anti-cancer agent is administered at a concentration of about 1 μM, about 2 μM, about 5 μM, about 10 μM, about 15 μM, about 20 μM, about 25 μM, about 30 μM, about 35 μM, about 40 μM, about 45 μM or about 50 μM.

In embodiments, the type I PRMT inhibitor has the chemical structure of formula:

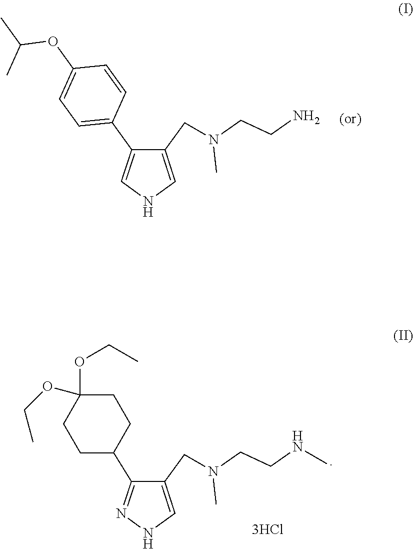 [SMILES: CC(C)Oc1ccc(-c2c[nH]cc2CN(C)CCN)cc1 ; CCOC1(OCC)CCC(c2n[nH]cc2CN(C)CCNC)CC1]

In embodiments, the type I PRMT inhibitor has the chemical structure of formula (I). In embodiments, the type I PRMT inhibitor has the chemical structure of formula (II). In embodiments, the type I PRMT inhibitor is MS023 or GSK3368715. In embodiments, the type I PRMT inhibitor is MS203. In embodiments, the type I PRMT inhibitor is GSK3368715.

In embodiments, the type I PRMT inhibitor is administered at an amount well known in the art effective to inhibit PRMT-1. In embodiments, the type I PRMT inhibitor is administered at a concentration of 0.001 μM to 10 μM. In embodiments, the type I PRMT inhibitor is administered at a concentration of 0.01 μM to 10 μM. In embodiments, the type I PRMT inhibitor is administered at a concentration of 0.1 μM to 10 μM. In embodiments, the type I PRMT inhibitor is administered at a concentration of 1 μM to 10 μM. In embodiments, the type I PRMT inhibitor is administered at a concentration of 2 μM to 10 μM. In embodiments, the type I PRMT inhibitor is administered at a concentration of 3 μM to 10 μM. In embodiments, the type I PRMT inhibitor is administered at a concentration of 4 μM to 10 μM. In embodiments, the type I PRMT inhibitor is administered at a concentration of 5 μM to 10 μM. In embodiments, the type I PRMT inhibitor is administered at a concentration of 6 μM to 10 μM. In embodiments, the type I PRMT inhibitor is administered at a concentration of 7 μM to 10 μM. In embodiments, the type I PRMT inhibitor is administered at a concentration of 8 μM to 10 μM. In embodiments, the type I PRMT inhibitor is administered at a concentration of 9 μM to 10 μM.

In embodiments, the type I PRMT inhibitor is administered at a concentration of 0.001 μM to 9 μM. In embodiments, the type I PRMT inhibitor is administered at a concentration of 0.001 μM to 8 μM. In embodiments, the type I PRMT inhibitor is administered at a concentration of 0.001 μM to 7 μM. In embodiments, the type I PRMT inhibitor is administered at a concentration of 0.001 μM to 6 μM. In embodiments, the type I PRMT inhibitor is administered at a concentration of 0.001 μM to 5 μM. In embodiments, the type I PRMT inhibitor is administered at a concentration of 0.001 μM to 4 μM. In embodiments, the type I PRMT inhibitor is administered at a concentration of 0.001 μM to 3 μM. In embodiments, the type I PRMT inhibitor is administered at a concentration of 0.001 μM to 2 μM. In embodiments, the type I PRMT inhibitor is administered at a concentration of 0.001 μM to 1 μM. In embodiments, the type I PRMT inhibitor is administered at a concentration of 0.001 μM to 0.1 μM. In embodiments, the type I PRMT inhibitor is administered at a concentration of 0.001 μM to 0.01 μM. In embodiments, the type I PRMT inhibitor is administered at a concentration of 0.001 μM, 0.01 μM, 0.1 μM, 1 μM, 2 μM, 3 μM, 4 μM, 5 μM, 6 μM, 7 μM, 8 μM, 9 μM or 10 μM.

In embodiments, the type I PRMT inhibitor is administered at a concentration of about 0.001 μM to about 10 μM. In embodiments, the type I PRMT inhibitor is administered at a concentration of about 0.01 μM to about 10 μM. In embodiments, the type I PRMT inhibitor is administered at a concentration of about 0.1 μM to about 10 μM. In embodiments, the type I PRMT inhibitor is administered at a concentration of about 1 μM to about 10 μM. In embodiments, the type I PRMT inhibitor is administered at a concentration of about 2 μM to about 10 μM. In embodiments, the type I PRMT inhibitor is administered at a concentration of about 3 μM to about 10 μM. In embodiments, the type I PRMT inhibitor is administered at a concentration of about 4 μM to about 10 μM. In embodiments, the type I PRMT inhibitor is administered at a concentration of about 5 μM to about 10 μM. In embodiments, the type I PRMT inhibitor is administered at a concentration of about 6 μM to about 10 μM. In embodiments, the type I PRMT inhibitor is administered at a concentration of about 7 μM to about 10 μM. In embodiments, the type I PRMT inhibitor is administered at a concentration of about 8 μM to about 10 μM. In embodiments, the type I PRMT inhibitor is administered at a concentration of about 9 μM to about 10 μM.

In embodiments, the type I PRMT inhibitor is administered at a concentration of about 0.001 μM to about 9 μM. In embodiments, the type I PRMT inhibitor is administered at a concentration of about 0.001 μM to about 8 μM. In embodiments, the type I PRMT inhibitor is administered at a concentration of about 0.001 μM to about 7 μM. In embodiments, the type I PRMT inhibitor is administered at a concentration of about 0.001 μM to about 6 μM. In embodiments, the type I PRMT inhibitor is administered at a concentration of about 0.001 μM to about 5 μM. In embodiments, the type I PRMT inhibitor is administered at a concentration of about 0.001 μM to about 4 μM. In embodiments, the type I PRMT inhibitor is administered at a concentration of about 0.001 μM to about 3 μM. In embodiments, the type I PRMT inhibitor is administered at a concentration of about 0.001 μM to about 2 μM. In embodiments, the type I PRMT inhibitor is administered at a concentration of about 0.001 μM to about 1 μM. In embodiments, the type I PRMT inhibitor is administered at a concentration of about 0.001 μM to about 0.1 μM. In embodiments, the type I PRMT inhibitor is administered at a concentration of about 0.001 μM to about 0.01 μM. In embodiments, the type I PRMT inhibitor is administered at a concentration of about 0.001 μM, about 0.01 μM, about 0.1 μM, about 1 μM, about 2 μM, about 3 μM, about 4 μM, about 5 μM, about 6 μM, about 7 μM, about 8 μM, about 9 μM or about 10 μM.

In embodiments, the cancer is lung cancer, colon cancer, kidney cancer, brain cancer, breast cancer, and/or pancreatic cancer. In embodiments, the cancer is lung cancer, colon cancer, kidney cancer, brain cancer, breast cancer, and pancreatic cancer. In embodiments, the cancer is lung cancer, colon cancer, kidney cancer, brain cancer, breast cancer, or pancreatic cancer. In embodiments, the cancer is lung cancer. In embodiments, the cancer is colon cancer. In embodiments, the cancer is kidney cancer. In embodiments, the cancer is brain cancer. In embodiments, the cancer is breast cancer. In embodiments, the cancer is pancreatic cancer. In embodiments, the cancer is a cancer selected from lung cancer, colon cancer, kidney cancer, brain cancer, breast cancer, and pancreatic cancer.

In embodiments, the subject has elevated IRF1 activity, elevated SOCS1 activity, elevated APOL1 activity, elevated B2M activity, elevated GBP activity, and/or elevated RNF213 activity. In embodiments, the subject has elevated IRF1 activity. In embodiments, the subject has elevated SOCS1 activity. In embodiments, the subject has elevated APOL1 activity. In embodiments, the subject has elevated B2M activity. In embodiments, the subject has elevated GBP activity. In embodiments, the subject has elevated RNF213 activity. In embodiments, the subject has elevated IRF1 activity, elevated SOCS1 activity, elevated APOL1 activity, elevated B2M activity, elevated GBP activity, and elevated RNF213 activity. In embodiments, the subject has elevated IRF1 activity, elevated SOCS1 activity, elevated APOL1 activity, elevated B2M activity, elevated GBP activity, or elevated RNF213 activity. In embodiments, the subject has elevated activity selected from IRF1 activity, elevated SOCS1 activity, elevated APOL1 activity, elevated B2M activity, elevated GBP activity, and elevated RNF213 activity.

In embodiments, the subject has elevated IRF1 activity, elevated SOCS1 activity, elevated APOL1 activity, elevated B2M activity, elevated GBP activity, and/or elevated RNF213 activity relative to a standard control. In embodiments, the subject has elevated IRF1 activity relative to a standard control. In embodiments, the subject has elevated SOCS1 activity relative to a standard control. In embodiments, the subject has elevated APOL1 activity relative to a standard control. In embodiments, the subject has elevated B2M activity relative to a standard control. In embodiments, the subject has elevated GBP activity relative to a standard control. In embodiments, the subject has elevated RNF213 activity relative to a standard control. In embodiments, the subject has elevated IRF1 activity, elevated SOCS1 activity, elevated APOL1 activity, elevated B2M activity, elevated GBP activity, and elevated RNF213 activity relative to a standard control. In embodiments, the subject has elevated IRF1 activity, elevated SOCS1 activity, elevated APOL1 activity, elevated B2M activity, elevated GBP activity, or elevated RNF213 activity relative to a standard control. In embodiments, the subject has elevated activity relative to a standard control the elevated activity selected from elevated IRF1 activity, elevated SOCS1 activity, elevated APOL1 activity, elevated B2M activity, elevated GBP activity, and elevated RNF213 activity.

In embodiments, the subject has elevated interferon alpha (IFNα) levels, interferon gamma (IFNγ) levels, epidermal growth factor (EGF) levels, platelet derived growth factor (PDGF) levels, and/or interleukin 6 (IL6) levels. In embodiments, the subject has elevated IFNα levels. In embodiments, an increased level of IFNα results in the increase of IFNα stimulated genes. In embodiments, the subject has elevated IFNγ levels. In embodiments, an increased level of IFNγ results in the increase of IFNγ stimulated genes. In embodiments, the subject has elevated EGF levels. In embodiments, an increased level of EGF results in the increase of EGF stimulated genes. In embodiments, the subject has elevated PDGF levels. In embodiments, an increased level of PDGF results in the increase of PDGF stimulated genes. In embodiments, the subject has elevated IL6 levels. In embodiments, an increased level of IL6 results in the increase of IL6 stimulated genes. In embodiments, the subject has elevated IFNα levels, IFNγ levels, EGF levels, PDGF levels, and IL6 levels. In embodiments, the subject has elevated IFNα levels, IFNγ levels, EGF levels, PDGF levels, or IL6 levels. In embodiments, the subject has elevated levels selected from IFNα levels, IFNγ levels, EGF levels, PDGF levels, and IL6 levels.

In embodiments, the subject has elevated interferon alpha (IFNα) levels, interferon gamma (IFNγ) levels, epidermal growth factor (EGF) levels, platelet derived growth factor (PDGF) levels, and/or interleukin 6 (IL6) levels relative to a standard control. In embodiments, the subject has elevated IFNα levels relative to a standard control. In embodiments, an increased level of IFNα results in the increase of IFNα stimulated genes relative to a standard control. In embodiments, the subject has elevated IFNγ levels relative to a standard control. In embodiments, an increased level of IFNγ results in the increase of IFNγ stimulated genes relative to a standard control. In embodiments, the subject has elevated EGF levels relative to a standard control. In embodiments, an increased level of EGF results in the increase of EGF stimulated genes relative to a standard control. In embodiments, the subject has elevated PDGF levels relative to a standard control. In embodiments, an increased level of PDGF results in the increase of PDGF stimulated genes relative to a standard control. In embodiments, the subject has elevated IL6 levels relative to a standard control. In embodiments, an increased level of IL6 results in the increase of IL6 stimulated genes relative to a standard control. In embodiments, the subject has elevated IFNα levels, IFNγ levels, EGF levels, PDGF levels, and IL6 levels relative to a standard control. In embodiments, the subject has elevated IFNα levels, IFNγ levels, EGF levels, PDGF levels, or IL6 levels relative to a standard control. In embodiments, the subject has elevated levels relative to a standard control the elevated levels selected from IFNα levels, IFNγ levels, EGF levels, PDGF levels, and IL6 levels.

In embodiments, the subject has a STAT1 activating genetic mutation. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and/or a SOCS1 amplification. In embodiments, the STAT1 activating genetic mutation is a JAK1/2 mutation. In embodiments, the STAT1 activating genetic mutation is an IFNGR1/2 mutation. In embodiments, the STAT1 activating genetic mutation is a B2M mutation. In embodiments, the STAT1 activating genetic mutation is a 9p21.33 disruption. In embodiments, the STAT1 activating genetic mutation is a CDKN2A deletion. In embodiments, the STAT1 activating genetic mutation is a 5q deletion. In embodiments, the STAT1 activating genetic mutation is an IRF1 inactivating rearrangement. In embodiments, the STAT1 activating genetic mutation is an IRF1 deletion. In embodiments, the STAT1 activating genetic mutation is a SOCS1 mutation. In embodiments, the STAT1 activating genetic mutation is a SOCS1 amplification. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and a SOCS1 amplification. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification. In embodiments, the STAT1 activating genetic mutation is a mutation selected from a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and a SOCS1 amplification.

In embodiments, the subject includes a tumor, the tumor including CD4+ T cells, CD8+ T cells, macrophages, neutrophils, and/or NK cells. In embodiments, the subject includes a tumor including CD4+ T cells. In embodiments, the subject includes a tumor including CD8+ T cells. In embodiments, the subject includes a tumor including macrophages. In embodiments, the subject includes a tumor including neutrophils. In embodiments, the subject includes a tumor including NK cells. In embodiments, the subject includes a tumor, the tumor including CD4+ T cells, CD8+ T cells, macrophages, neutrophils, and NK cells. In embodiments, the subject includes a tumor, the tumor including CD4+ T cells, CD8+ T cells, macrophages, neutrophils, or NK cells.

In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, and/or 4-1BBL. In embodiments, the subject includes a tumor, the tumor including PD-1. In embodiments, the subject includes a tumor, the tumor including PD-L1/2. In embodiments, the subject includes a tumor, the tumor including CD155. In embodiments, the subject includes a tumor, the tumor including CD80/86. In embodiments, the subject includes a tumor, the tumor including CD28. In embodiments, the subject includes a tumor, the tumor including CTLA-4. In embodiments, the subject includes a tumor, the tumor including galectin-9. In embodiments, the subject includes a tumor, the tumor including TIM3. In embodiments, the subject includes a tumor, the tumor including Siglec-15. In embodiments, the subject includes a tumor, the tumor including ICOS. In embodiments, the subject includes a tumor, the tumor including ICOS-L. In embodiments, the subject includes a tumor, the tumor including CD47. In embodiments, the subject includes a tumor, the tumor including CD70. In embodiments, the subject includes a tumor, the tumor including 4-1BBL. In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, and 4-1BBL. In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, or 4-1BBL.

In embodiments, the tumor includes cells expressing PD-1, PD-L1/2, CD155, CD80/86, CD28, CTLA-4, galectin-9, TIM3, Siglec-15, ICOS, ICOS-L, CD47, CD70, and/or 4-1BBL. In embodiments, the tumor includes cells expressing PD-1. In embodiments, the tumor includes cells expressing PD-L1/2. In embodiments, the tumor includes cells expressing CD155. In embodiments, the tumor includes cells expressing CD80/86. In embodiments, the tumor includes cells expressing CD28. In embodiments, the tumor includes cells expressing CTLA-4. In embodiments, the tumor includes cells expressing galectin-9. In embodiments, the tumor includes cells expressing TIM3. In embodiments, the tumor includes cells expressing Siglec-15. In embodiments, the tumor includes cells expressing ICOS. In embodiments, the tumor includes cells expressing ICOS-L. In embodiments, the tumor includes cells expressing CD47. In embodiments, the tumor includes cells expressing CD70. In embodiments, the tumor includes cells expressing 4-1BBL. In embodiments, the tumor includes cells expressing PD-1, PD-L1/2, CD155, CD80/86, CD28, CTLA-4, galectin-9, TIM3, Siglec-15, ICOS, ICOS-L, CD47, CD70, and 4-1BBL. In embodiments, the tumor includes cells expressing PD-1, PD-L1/2, CD155, CD80/86, CD28, CTLA-4, galectin-9, TIM3, Siglec-15, ICOS, ICOS-L, CD47, CD70, or 4-1BBL. In further embodiments, the cells are non-tumor cells. In further embodiments, the cells are tumor cells.

In embodiments, the subject includes a tumor, the tumor including TAP, B2M, CIITA, HLA-DR, and/or HLA-DMA. In embodiments, the subject includes a tumor, the tumor including TAP. In embodiments, the subject includes a tumor, the tumor including B2M. In embodiments, the subject includes a tumor, the tumor including CIITA. In embodiments, the subject includes a tumor, the tumor including HLA-DR. In embodiments, the subject includes a tumor, the tumor including HLA-DMA. In embodiments, the subject includes a tumor, the tumor including TAP, B2M, CIITA, HLA-DR, and HLA-DMA. In embodiments, the subject includes a tumor, the tumor including TAP, B2M, CIITA, HLA-DR, or HLA-DMA.

In embodiments, the tumor includes cells expressing TAP, B2M, CIITA, HLA-DR, and/or HLA-DMA. In embodiments, the tumor includes cells expressing TAP. In embodiments, the tumor includes cells expressing B2M. In embodiments, the tumor includes cells expressing CIITA. In embodiments, the tumor includes cells expressing HLA-DR. In embodiments, the tumor includes cells expressing HLA-DMA. In embodiments, the tumor includes cells expressing TAP, B2M, CIITA, HLA-DR, and HLA-DMA. In embodiments, the tumor includes cells expressing TAP, B2M, CIITA, HLA-DR, or HLA-DMA. In further embodiments, the cells are non-tumor cells. In further embodiments, the cells are tumor cells.

In an aspect is provided a method of treating cancer in a subject, the method including administering a therapeutically effective amount of a type I PRMT inhibitor to the subject, wherein the subject has been previously treated with a STAT1 activating compound.

In an aspect is provided a method of treating cancer in a subject, the method including administering a therapeutically effective amount of a type I PRMT inhibitor and a STAT1 activating compound to the subject.

As defined herein, the term “activation”, “activate”, “activating”, “activator” and the like in reference to a protein-activator interaction means positively affecting (e.g. increasing) the activity or function of the protein relative to the activity or function of the protein in the absence of the activator. In embodiments activation means positively affecting (e.g. increasing) the concentration or levels of the protein relative to the concentration or level of the protein in the absence of the activator. In embodiments, activation refers to an increase in the activity of a particular protein target. In embodiments, activation refers to activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). In embodiments, activation may include, at least in part, partially or totally increasing stimulation, increasing or enabling activation, or activating, sensitizing, or up-regulating signal transduction or enzymatic activity or the amount of a protein

The terms “STAT1 activator,” “STAT1 upregulator,” “STAT1 activating compound,” etc. refer to a substance capable of detectably increasing the expression or activity of a STAT1 gene or a STAT1 protein. The STAT1 activating compound can increase expression or activity by 10%, 20%, 30%, 40%, 50%, 60%, 70%, 80%, 90% or more in comparison to a control in the absence of the activator. In certain instances, STAT1 expression or STAT1 activity is 1.5-fold, 2-fold, 3-fold, 4-fold, 5-fold, 10-fold or higher than the STAT1 expression or STAT1 activity in the absence of the STAT1 activating compound.

In embodiments, the STAT1 activating compound is an anti-cancer agent. In embodiments, the anti-cancer agent is erlotinib, osimertinib, carboplatin and/or gemcitabine. In embodiments, the anti-cancer agent is erlotinib. In embodiments, the anti-cancer agent is osimertinib. In embodiments, the anti-cancer agent is carboplatin. In embodiments, the anti-cancer agent is gemcitabine. In embodiments, the anti-cancer agent is erlotinib, osimertinib, carboplatin and gemcitabine. In embodiments, the anti-cancer agent is erlotinib, osimertinib, carboplatin or gemcitabine. In embodiments, the anti-cancer agent is an anti-cancer agent selected from erlotinib, osimertinib, carboplatin, and gemcitabine.

In embodiments, the type I PRMT inhibitor has the chemical structure of formula (I) or formula (II). In embodiments, the type I PRMT inhibitor has the chemical structure of formula (I). In embodiments, the type I PRMT inhibitor has the chemical structure of formula (II). In embodiments, the type I PRMT inhibitor is MS023 or GSK3368715. In embodiments, the type I PRMT inhibitor is MS203. In embodiments, the type I PRMT inhibitor is GSK3368715.

In embodiments, the cancer is lung cancer, colon cancer, kidney cancer, brain cancer, breast cancer, and/or pancreatic cancer. In embodiments, the cancer is lung cancer. In embodiments, the cancer is colon cancer. In embodiments, the cancer is kidney cancer. In embodiments, the cancer is brain cancer. In embodiments, the cancer is breast cancer. In embodiments, the cancer is pancreatic cancer. In embodiments, the cancer is lung cancer, colon cancer, kidney cancer, brain cancer, breast cancer, and pancreatic cancer. In embodiments, the cancer is lung cancer, colon cancer, kidney cancer, brain cancer, breast cancer, or pancreatic cancer. In embodiments, the cancer is a cancer selected from lung cancer, colon cancer, kidney cancer, brain cancer, breast cancer, and pancreatic cancer.

In an aspect is provided a method of treating cancer in a subject in need thereof, the method including: (i) detecting an elevated STAT1 activity in a subject; and (ii) administering a therapeutically effective amount of a type I PRMT inhibitor to the subject.

In an aspect is provided a method of treating cancer in a subject in need thereof, the method including: (i) detecting an elevated STAT1 activity in a subject; and (ii) administering a therapeutically effective amount of an anti-cancer agent to the subject.

In an aspect is provided a method of treating cancer in a subject in need thereof, the method including: (i) detecting a STAT1 activity in a subject; and (ii) administering a therapeutically effective amount of a STAT1 activating compound to the subject.

In embodiments, the method further includes administering to the subject an effective amount of a type I PRMT inhibitor. In embodiments, the type I PRMT inhibitor has the chemical structure of formula (I) or formula (II). In embodiments, the type I PRMT inhibitor has the chemical structure of formula (I). In embodiments, the type I PRMT inhibitor has the chemical structure of formula (II). In embodiments, the type I PRMT inhibitor is MS023 or GSK3368715. In embodiments, the type I PRMT inhibitor is MS203. In embodiments, the type I PRMT inhibitor is GSK3368715.

In embodiments, the STAT1 activating compound is an anti-cancer agent. In embodiments, the anti-cancer agent is erlotinib, osimertinib, carboplatin and/or gemcitabine. In embodiments, the anti-cancer agent is erlotinib. In embodiments, the anti-cancer agent is osimertinib. In embodiments, the anti-cancer agent is carboplatin. In embodiments, the anti-cancer agent is gemcitabine. In embodiments, the anti-cancer agent is erlotinib, osimertinib, carboplatin and gemcitabine. In embodiments, the anti-cancer agent is erlotinib, osimertinib, carboplatin or gemcitabine. In embodiments, the anti-cancer agent is an anti-cancer agent selected from erlotinib, osimertinib, carboplatin, and gemcitabine.

In embodiments, the subject is or has been treated with an anti-cancer agent. In embodiments, the anti-cancer agent is erlotinib, osimertinib, carboplatin and/or gemcitabine. In embodiments, the anti-cancer agent is erlotinib. In embodiments, the anti-cancer agent is osimertinib. In embodiments, the anti-cancer agent is carboplatin. In embodiments, the anti-cancer agent is gemcitabine. In embodiments, the anti-cancer agent is erlotinib, osimertinib, carboplatin and gemcitabine. In embodiments, the anti-cancer agent is erlotinib, osimertinib, carboplatin or gemcitabine. In embodiments, the anti-cancer agent is an anti-cancer agent selected from erlotinib, osimertinib, carboplatin, and gemcitabine.

In embodiments, the cancer is lung cancer, colon cancer, kidney cancer, brain cancer, breast cancer, and/or pancreatic cancer. In embodiments, the cancer is lung cancer. In embodiments, the cancer is colon cancer. In embodiments, the cancer is kidney cancer. In embodiments, the cancer is brain cancer. In embodiments, the cancer is breast cancer. In embodiments, the cancer is pancreatic cancer. In embodiments, the cancer is lung cancer, colon cancer, kidney cancer, brain cancer, breast cancer, and pancreatic cancer. In embodiments, the cancer is lung cancer, colon cancer, kidney cancer, brain cancer, breast cancer, or pancreatic cancer. In embodiments, the cancer is a cancer selected from lung cancer, colon cancer, kidney cancer, brain cancer, breast cancer, and pancreatic cancer.

In embodiments, the subject has elevated IRF1 activity, elevated SOCS1 activity, elevated APOL1 activity, elevated B2M activity, elevated GBP activity, and/or elevated RNF213 activity. In embodiments, the subject has elevated IRF1 activity. In embodiments, the subject has elevated SOCS1 activity. In embodiments, the subject has elevated APOL1 activity. In embodiments, the subject has elevated B2M activity. In embodiments, the subject has elevated GBP activity. In embodiments, the subject has elevated RNF213 activity. In embodiments, the subject has elevated IRF1 activity, elevated SOCS1 activity, elevated APOL1 activity, elevated B2M activity, elevated GBP activity, and elevated RNF213 activity. In embodiments, the subject has elevated IRF1 activity, elevated SOCS1 activity, elevated APOL1 activity, elevated B2M activity, elevated GBP activity, or elevated RNF213 activity. In embodiments, the subject has an elevated activity selected from IRF1 activity, elevated SOCS1 activity, elevated APOL1 activity, elevated B2M activity, elevated GBP activity, and elevated RNF213 activity.

In embodiments, the subject has elevated IRF1 activity, elevated SOCS1 activity, elevated APOL1 activity, elevated B2M activity, elevated GBP activity, and/or elevated RNF213 activity relative to a standard control In embodiments, the subject has elevated IRF1 activity relative to a standard control. In embodiments, the subject has elevated SOCS1 activity relative to a standard control. In embodiments, the subject has elevated APOL1 activity relative to a standard control. In embodiments, the subject has elevated B2M activity relative to a standard control. In embodiments, the subject has elevated GBP activity relative to a standard control. In embodiments, the subject has elevated RNF213 activity relative to a standard control. In embodiments, the subject has elevated IRF1 activity, elevated SOCS1 activity, elevated APOL1 activity, elevated B2M activity, elevated GBP activity, and elevated RNF213 activity relative to a standard control. In embodiments, the subject has elevated IRF1 activity, elevated SOCS1 activity, elevated APOL1 activity, elevated B2M activity, elevated GBP activity, or elevated RNF213 activity relative to a standard control. In embodiments, the subject has an elevated activity relative to a standard control the elevated activity selected from elevated IRF1 activity, elevated SOCS1 activity, elevated APOL1 activity, elevated B2M activity, elevated GBP activity, and elevated RNF213 activity.

In embodiments, the subject has elevated interferon alpha (IFNα) levels, interferon gamma (IFNγ) levels, epidermal growth factor (EGF) levels, platelet derived growth factor (PDGF) levels, and/or interleukin 6 (IL6) levels. In embodiments, the subject has elevated IFNα levels. In embodiments, an increased level of IFNα results in the increase of IFNα stimulated genes. In embodiments, the subject has elevated IFNγ levels. In embodiments, an increased level of IFNγ results in the increase of IFNγ stimulated genes. In embodiments, the subject has elevated EGF levels. In embodiments, an increased level of EGF results in the increase of EGF stimulated genes. In embodiments, the subject has elevated PDGF levels. In embodiments, an increased level of PDGF results in the increase of PDGF stimulated genes. In embodiments, the subject has elevated IL6 levels. In embodiments, an increased level of IL6 results in the increase of IL6 stimulated genes. In embodiments, the subject has elevated IFNα levels, IFNγ levels, EGF levels, PDGF levels, and IL6 levels. In embodiments, the subject has elevated IFNα levels, IFNγ levels, EGF levels, PDGF levels, or IL6 levels. In embodiments, the subject has elevated levels selected from IFNα levels, IFNγ levels, EGF levels, PDGF levels, and IL6 levels.

In embodiments, the subject has elevated interferon alpha (IFNα) levels, interferon gamma (IFNγ) levels, epidermal growth factor (EGF) levels, platelet derived growth factor (PDGF) levels, and/or interleukin 6 (IL6) levels relative to a standard control. In embodiments, the subject has elevated IFNα levels relative to a standard control. In embodiments, an increased level of IFNα results in the increase of IFNα stimulated genes relative to a standard control. In embodiments, the subject has elevated IFNγ levels relative to a standard control. In embodiments, an increased level of IFNγ results in the increase of IFNγ stimulated genes relative to a standard control. In embodiments, the subject has elevated EGF levels relative to a standard control. In embodiments, an increased level of EGF results in the increase of EGF stimulated genes relative to a standard control. In embodiments, the subject has elevated PDGF levels relative to a standard control. In embodiments, an increased level of PDGF results in the increase of PDGF stimulated genes relative to a standard control. In embodiments, the subject has elevated IL6 levels relative to a standard control. In embodiments, an increased level of IL6 results in the increase of IL6 stimulated genes relative to a standard control. In embodiments, the subject has elevated IFNα levels, IFNγ levels, EGF levels, PDGF levels, and IL6 levels relative to a standard control. In embodiments, the subject has elevated IFNα levels, IFNγ levels, EGF levels, PDGF levels, or IL6 levels relative to a standard control. In embodiments, the subject has elevated levels relative to a standard control the elevated levels selected from IFNα levels, IFNγ levels, EGF levels, PDGF levels, and IL6 levels.

In embodiments, the subject has a STAT1 activating genetic mutation. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and/or a SOCS1 amplification. In embodiments, the STAT1 activating genetic mutation is a JAK1/2 mutation. In embodiments, the STAT1 activating genetic mutation is an IFNGR1/2 mutation. In embodiments, the STAT1 activating genetic mutation is a B2M mutation. In embodiments, the STAT1 activating genetic mutation is a 9p21.33 disruption. In embodiments, the STAT1 activating genetic mutation is a CDKN2A deletion. In embodiments, the STAT1 activating genetic mutation is a 5q deletion. In embodiments, the STAT1 activating genetic mutation is an IRF1 inactivating rearrangement. In embodiments, the STAT1 activating genetic mutation is an IRF1 deletion. In embodiments, the STAT1 activating genetic mutation is a SOCS1 mutation. In embodiments, the STAT1 activating genetic mutation is a SOCS1 amplification. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and a SOCS1 amplification. In embodiments, the STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification. In embodiments, the STAT1 activating genetic mutation is a mutation selected from a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation and a SOCS1 amplification.

In embodiments, the subject includes a tumor, the tumor including CD4+ T cells, CD8+ T cells, macrophages, neutrophils, and/or NK cells. In embodiments, the subject includes a tumor including CD4+ T cells. In embodiments, the subject includes a tumor including CD8+ T cells. In embodiments, the subject includes a tumor including macrophages. In embodiments, the subject includes a tumor including neutrophils. In embodiments, the subject includes a tumor including NK cells. In embodiments, the subject includes a tumor, the tumor including CD4+ T cells, CD8+ T cells, macrophages, neutrophils, and NK cells. In embodiments, the subject includes a tumor, the tumor including CD4+ T cells, CD8+ T cells, macrophages, neutrophils, or NK cells.

In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, and/or 4-1BBL. In embodiments, the subject includes a tumor, the tumor including PD-1. In embodiments, the subject includes a tumor, the tumor including PD-L1/2. In embodiments, the subject includes a tumor, the tumor including CD155. In embodiments, the subject includes a tumor, the tumor including CD80/86. In embodiments, the subject includes a tumor, the tumor including CD28. In embodiments, the subject includes a tumor, the tumor including CTLA-4. In embodiments, the subject includes a tumor, the tumor including galectin-9. In embodiments, the subject includes a tumor, the tumor including TIM3. In embodiments, the subject includes a tumor, the tumor including Siglec-15. In embodiments, the subject includes a tumor, the tumor including ICOS. In embodiments, the subject includes a tumor, the tumor including ICOS-L. In embodiments, the subject includes a tumor, the tumor including CD47. In embodiments, the subject includes a tumor, the tumor including CD70. In embodiments, the subject includes a tumor, the tumor including 4-1BBL. In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, and 4-1BBL. In embodiments, the subject includes a tumor, the tumor including PD-1, PD-L1/2, CD155, CD80/86, CD28, CTLA-4, galectin-9, TIM3, Siglec-15, ICOS, ICOS-L, CD47, CD70, or 4-1BBL.

In embodiments, the tumor includes cells expressing PD-1, PD-L1/2, CD155, CD80/86, CD28, CTLA-4, galectin-9, TIM3, Siglec-15, ICOS, ICOS-L, CD47, CD70, and/or 4-1BBL. In embodiments, the tumor includes cells expressing PD-1. In embodiments, the tumor includes cells expressing PD-L1/2. In embodiments, the tumor includes cells expressing CD155. In embodiments, the tumor includes cells expressing CD80/86. In embodiments, the tumor includes cells expressing CD28. In embodiments, the tumor includes cells expressing CTLA-4. In embodiments, the tumor includes cells expressing galectin-9. In embodiments, the tumor includes cells expressing TIM3. In embodiments, the tumor includes cells expressing Siglec-15. In embodiments, the tumor includes cells expressing ICOS. In embodiments, the tumor includes cells expressing ICOS-L. In embodiments, the tumor includes cells expressing CD47. In embodiments, the tumor includes cells expressing CD70. In embodiments, the tumor includes cells expressing 4-1BBL. In embodiments, the tumor includes cells expressing PD-1, PD-L1/2, CD155, CD80/86, CD28, CTLA-4, galectin-9, TIM3, Siglec-15, ICOS, ICOS-L, CD47, CD70, and 4-1BBL. In embodiments, the tumor includes cells expressing PD-1, PD-L1/2, CD155, CD80/86, CD28, CTLA-4, galectin-9, TIM3, Siglec-15, ICOS, ICOS-L, CD47, CD70, or 4-1BBL. In further embodiments, the cells are non-tumor cells. In further embodiments, the cells are tumor cells.

In embodiments, the subject includes a tumor, the tumor including TAP, B2M, CIITA, HLA-DR, and/or HLA-DMA. In embodiments, the subject includes a tumor, the tumor including TAP. In embodiments, the subject includes a tumor, the tumor including B2M. In embodiments, the subject includes a tumor, the tumor including CIITA. In embodiments, the subject includes a tumor, the tumor including HLA-DR. In embodiments, the subject includes a tumor, the tumor including HLA-DMA. In embodiments, the subject includes a tumor, the tumor including TAP, B2M, CIITA, HLA-DR, and HLA-DMA. In embodiments, the subject includes a tumor, the tumor including TAP, B2M, CIITA, HLA-DR, or HLA-DMA.

In embodiments, the tumor includes cells expressing TAP, B2M, CIITA, HLA-DR, and/or HLA-DMA. In embodiments, the tumor includes cells expressing TAP. In embodiments, the tumor includes cells expressing B2M. In embodiments, the tumor includes cells expressing CIITA. In embodiments, the tumor includes cells expressing HLA-DR. In embodiments, the tumor includes cells expressing HLA-DMA. In embodiments, the tumor includes cells expressing TAP, B2M, CIITA, HLA-DR, and HLA-DMA. In embodiments, the tumor includes cells expressing TAP, B2M, CIITA, HLA-DR, or HLA-DMA. In further embodiments, the cells are non-tumor cells. In further embodiments, the cells are tumor cells.

### EXAMPLES

EGFR-targeting therapies are standard-of-care. However, relapse is almost inevitable due to residual cells that can survive the treatment. Our discoveries are: 1) PRMT antagonizes EGFR-targeted therapy in the presence of IFNg; 2) inhibition of PRMT sensitizes cells and diminishes residual disease for EGFR-targeted treatment; and 3) novel disease-relevant cellular assays to test PRMT drugs.

### Example 1: Definitions of “Signature of STAT1 Activation”

We defined a “signature of STAT1 activation” by a higher level of STAT1 or interferon-gamma expression, or a collection of downstream genes induced by STAT1 activation (e.g. IRF1, SOCS1, APOL1, B2M, GBPs, RNF213), or of these in combination with STAT1 or interferon-gamma expression as measured by mRNA or protein level from a tumor biopsy (as measured by bulk sample score or positive cell count) in comparison to a control sample. A control could be a matched sample from adjacent normal tissue or blood, a biopsy from a previous assessment (e.g. before and/or after first line treatment), or a sample or collections of “reference” levels from one or more healthy individual. Exemplary threshold levels of STAT1 could be 2, 2.5, 3, or more than 3-fold higher than control.

Alternatively, we defined a “signature of STAT1 activation” as genetic variations in STAT1 signaling molecules, for example JAK1/2 mutation, IFNGR1/2 mutation, B2M mutation, copy number loss of the interferon gene cluster due to 9p21.33 disruption, CDKN2A deletion, 5q deletion, IRF1 inactivating rearrangement and deletion, and SOCS1 mutation or amplification. These genetic variations included point mutations, gene truncations, deletions, amplifications, or gene fusions; the variations could be somatic mutations or germline mutations and could be heterozygous or homozygous.

Another alternative definition of “signature of STAT1 activation” relied on the infiltration of immune cells into the tumor or tumor microenvironment (for example the detection of CD4+ T cells, CD8+ T cells, macrophages, neutrophils, or NK cells in tumor biopsy samples), a detectable expression level of immune co-stimulatory molecules (for example the detection of PD-1, PD-L1/2, CD155, CD80/86, CD28, CTLA-4, galectin-9, TIM3, Siglec-15, ICOS, ICOS-L, CD47, CD70, or 4-1BBL expression in tumor biopsy samples), or a detectable expression level of antigen presentation molecules (for example the detection of TAP, B2M, CIITA, HLA-DR, or HLA-DMA expression in tumor biopsy samples), as measured by bulk sample score, positive cell count, or greater expression (e.g. 2-fold higher) in comparison to a control sample.

A tumor to be treated could be a tumor that was refractory to and relapsed from first line therapeutics, a treatment-naïve tumor, a localized tumor, or a metastatic tumor, a solid tumor, or a liquid tumor.

A treatment strategy could be administering a type I PRMT inhibitor to a tumor with signature of STAT1 activation, administering a type I PRMT inhibitor and an anti-cancer agent to a tumor with signature of STAT1 activation; administering a STAT1-activating modality and a type I PRMT inhibitor to a tumor with low or no detectable STAT1 activation (a STAT1-activating modality could be interferon-alpha, interferon-beta, interferon-gamma, immune checkpoint blockade, radiation, chemotherapy, or targeted therapy).

### Example 2: Inhibition of Type I PRMT Restrains STAT1 Expression and Eliminates Cancer Drug Tolerance

### Summary

Small populations of “drug-tolerant” cancer cells can survive drug treatment and serve as a reservoir for relapse^{1,2}. There is increasing evidence that cancer drug tolerance can emerge from non-genetic mechanisms³. However, what cell signaling states modulate the formation of drug tolerance, and how signaling-mediated drug tolerance can be eliminated are poorly understood. Here, we found that drug tolerance could be mediated by signal transducer and activator of transcription 1 (STAT1) transduced signaling—which itself could be activated by the tumor-immune microenvironment—and eliminated by inhibition of type I protein arginine methyltransferases (PRMTs). Mechanistic studies revealed that inhibition of type I PRMTs reduces protein, but not mRNA expression of STAT1. Further, poly(A)-binding protein 2 (PABP2) was found to be implicated as a functional link between type I PRMTs and STAT1: type I PRMTs directly methylated PABP2, and knockdown of PABP2 reduced STAT1 protein expression. Inhibition of type I PRMTs was observed to eliminate drug-tolerance for standard-of-care (SOC) drugs across multiple cancer models. Our studies identified type I PRMT inhibition as a novel vulnerability for eliminating STAT1 signaling-mediated drug tolerance.

### Introduction

Incomplete killing of cancer cells is a major challenge across most, if not all, cancer types and therapeutics^{4,5}. Mechanisms beyond genetic heterogeneity can contribute to incomplete killing⁶⁻¹¹. Evidence for non-genetic mechanisms includes the reversibility of the drug-tolerant state in models of cancer cell lines^{1,12} and the re-sensitization of cancers to initial therapeutics after treatment holidays in clinic^{1,14}. Eliminating non-genetic drug tolerance is an important goal, as tolerance has been observed for multiple types of cancers and diverse mechanisms of drugs. However, identifying the molecular logic underpinning drug tolerance is challenging: tolerance may arise from an interplay between environmental and cancer cell intrinsic signals; signaling pathways are notoriously interconnected and often pleiotropic; and epigenetic modifiers can influence hundreds, if not thousands, of molecular components. The ultimate goal of finding specific and actionable targets for eliminating tolerance within these combinatorial changes is daunting.

### Type I PRMT Inhibition Eliminates Drug Tolerance in the Presence of IFNγ Signaling

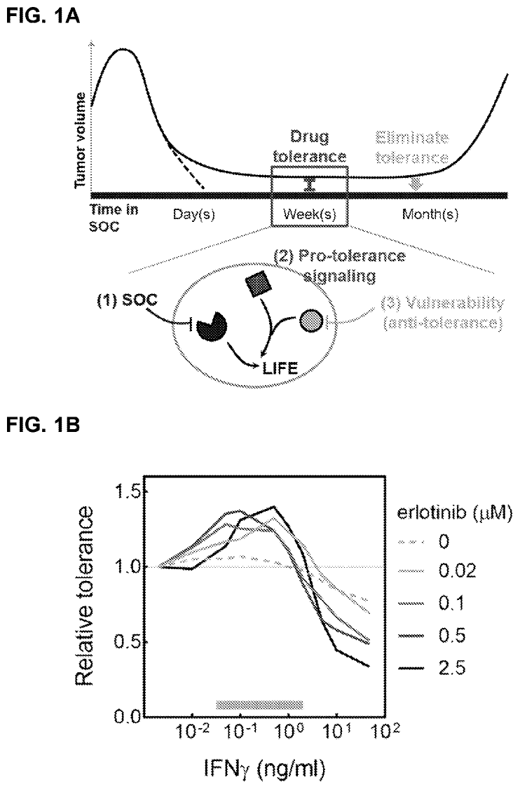

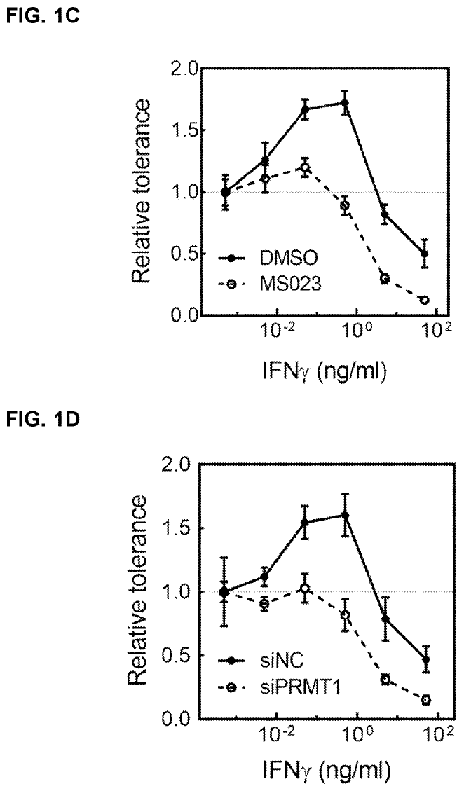

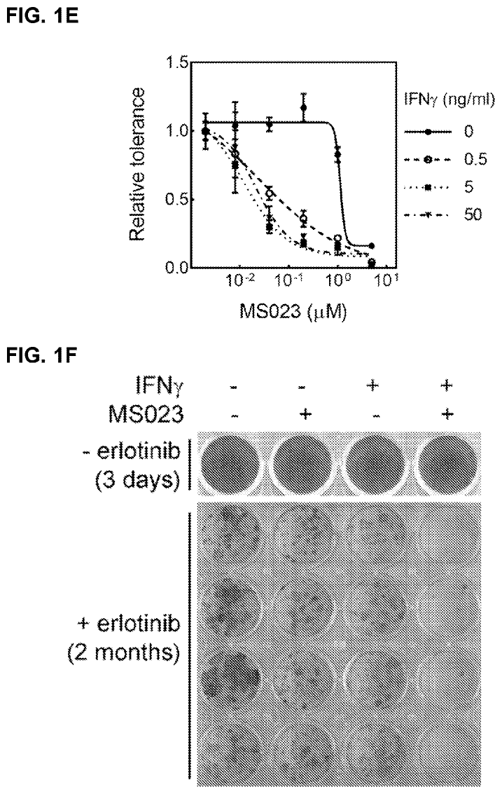

To investigate drug tolerance, we undertook a three-step approach (FIG. 1A). First, we established reproducible assays to monitor small subpopulations of cancer cells that survive sustained SOC drug treatment. We initially selected the well-established drug-tolerance model provided by the EGFR-mutant lung cancer cell line PC9 and EGFR inhibitor erlotinib^{1,2,6}. The effect of perturbations to drug tolerance was assessed by quantifying changes to the fraction of the small subpopulation of surviving (drug-tolerant) cells after approximately one week of continuous drug treatment.

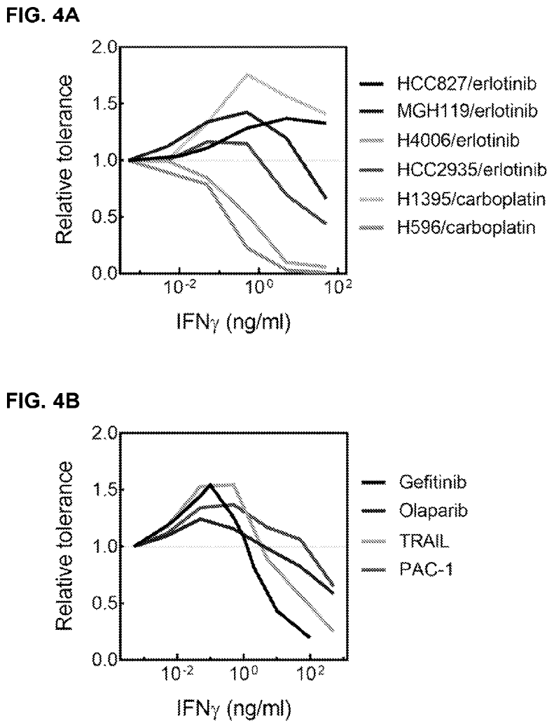

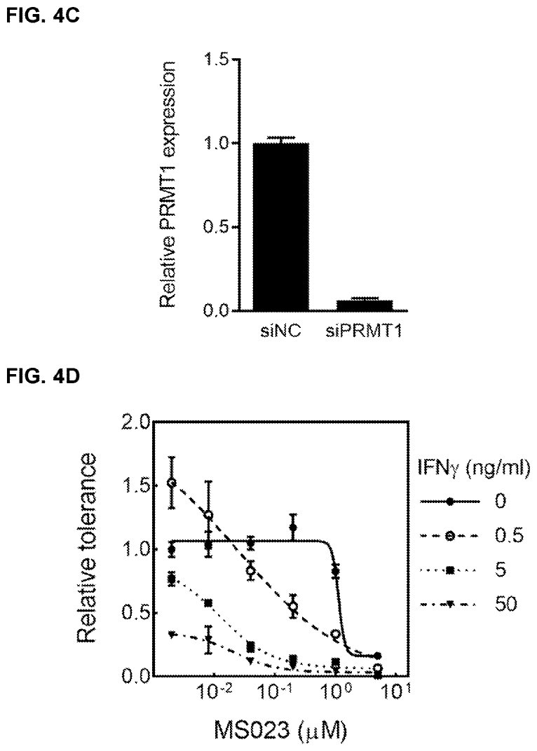

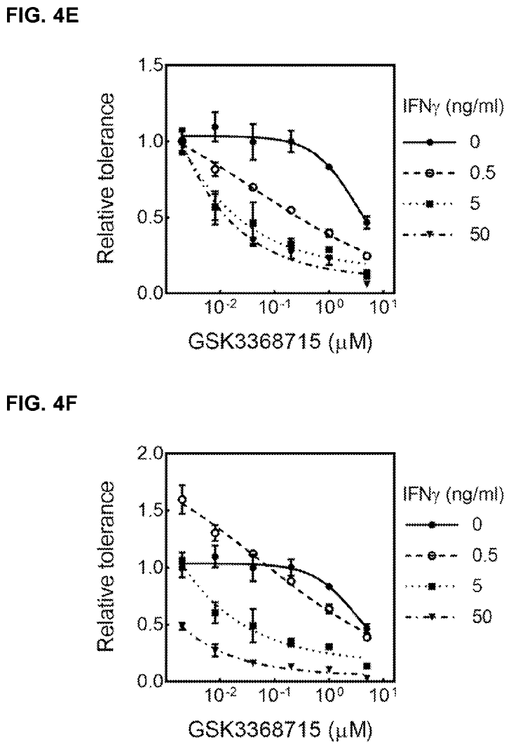

Second, we searched for cell-signaling inputs that affect drug tolerance. We made use of a collection of soluble factors and signaling molecules reported present in tumors or/and their microenvironments. A number of inputs strongly increase (e.g. fibroblast growth factor) or decrease (e.g. IGFR1 inhibitor) drug tolerance as expected^{1,15}. However, one of the most intriguing inputs was interferon gamma (IFNγ). Consistent with its anti-cancer role, IFNγ decreases drug tolerance at relatively high doses. Unexpectedly, relatively low doses of IFNγ increase tolerance in our PC9/erlotinib model (FIG. 1B) and, more generally, across other models of tolerance (FIGS. 4A and 4B). Recent studies have suggested a pro-survival role for IFNγ; specifically, an interferon-related DNA damage resistance signature (IRDS) has been associated with radiation-resistant cancer cells^{11,17}. Yet, how IFNγ signals—present in the tumor-immune microenvironment—could aid in the emergence of drug tolerance is unclear. Thus, IFNγ stimulation in our model provided an unexplored starting point to understand cell signaling that leads to the emergence of drug tolerance.

Third, we searched for perturbations that reduce IFNγ-stimulated drug tolerance within a collection of compounds of known biological functions and targets. We found that drug tolerance in the presence of IFNγ signaling was reduced by inhibition of type I PRMTs-enzymes that catalyze the transfer of methyl group from S-adenosyl methionine to the guanidine nitrogen atoms of arginine in their substrate protein. This result was observed for the pan-type I PRMT inhibitor MS023 across a wide range of IFNγ concentrations (FIG. 1C) and was subsequently confirmed by knockdown of PRMT1, the major isoform of type I PRMTs (FIG. 4C; FIG. 1D). The effect on reducing drug tolerance by type I PRMT inhibition was only observed in the presence of IFNγ stimulation as seen for MS023 (FIG. 1E; FIG. 4D) as well as the recently-developed pan-type I PRMT inhibitor GSK3368715 (FIGS. 4E-4F). Finally, a two-month co-treatment of MS023 with erlotinb durably reduced the expansion of drug-tolerant cells (referred to as persistors¹) in the context of IFNγ stimulation (FIG. 1F). Thus, type I PRMT inhibition is a vulnerability of IFNγ signaling-stimulated drug tolerance.

### STAT1 Provides Pro-Tolerance Signal and Type I PRMT Inhibition Restrains STAT1 Protein Synthesis

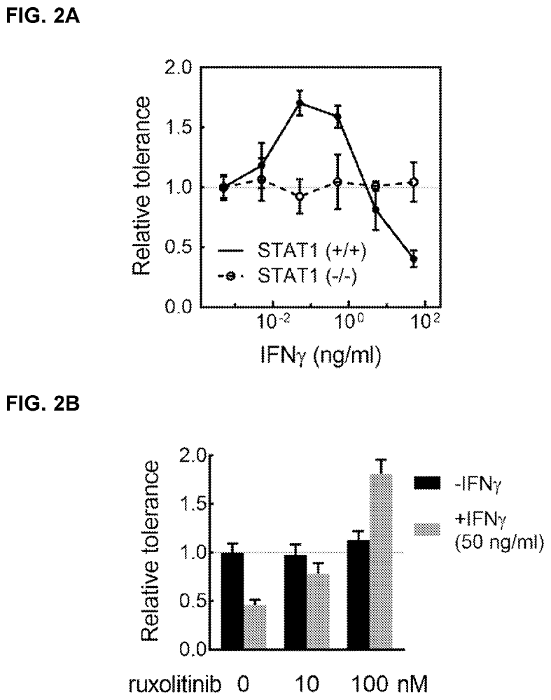

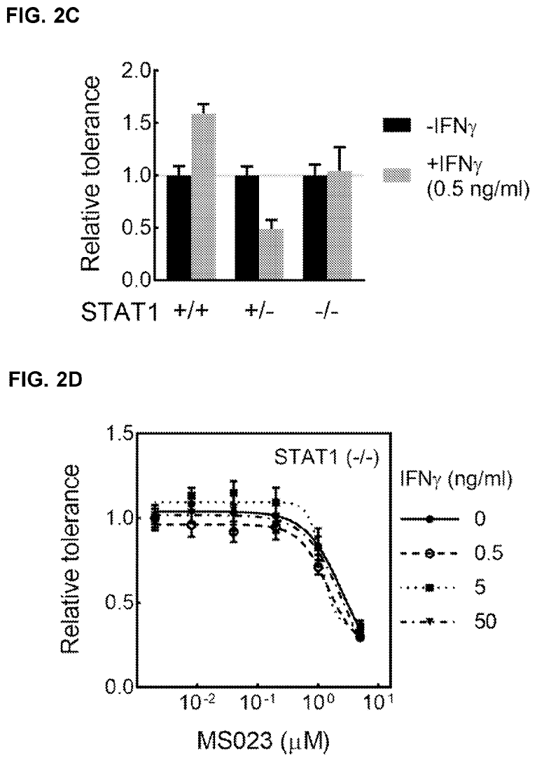

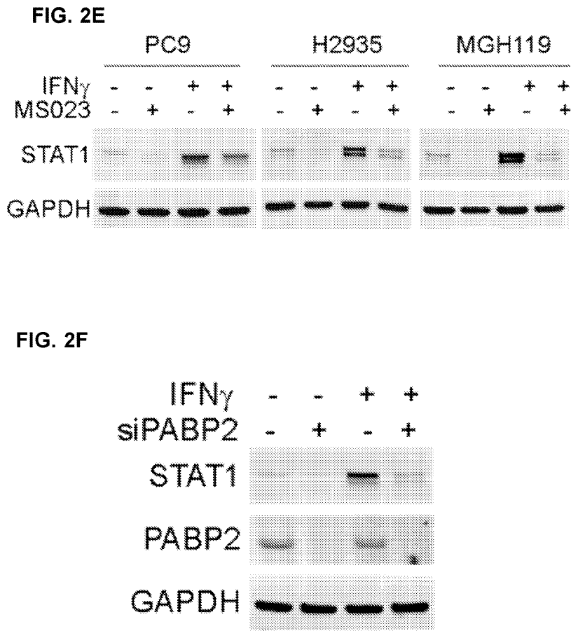

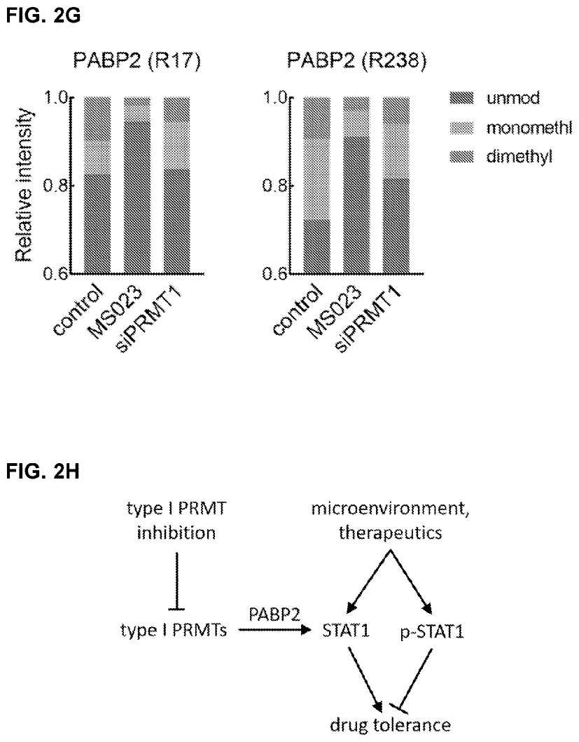

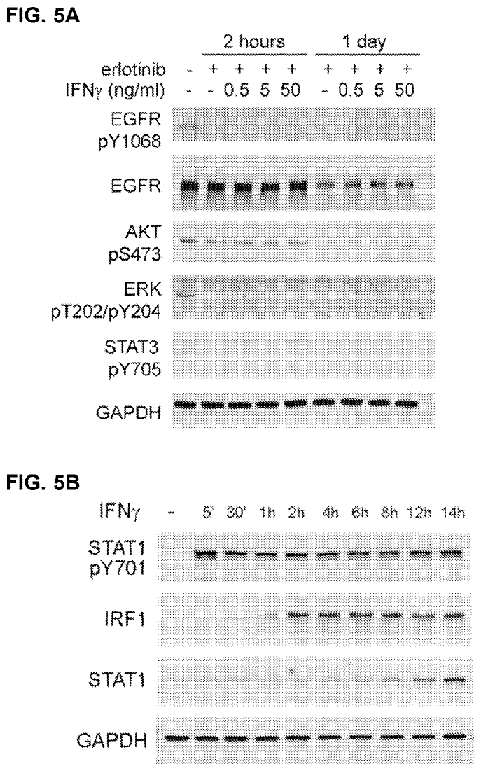

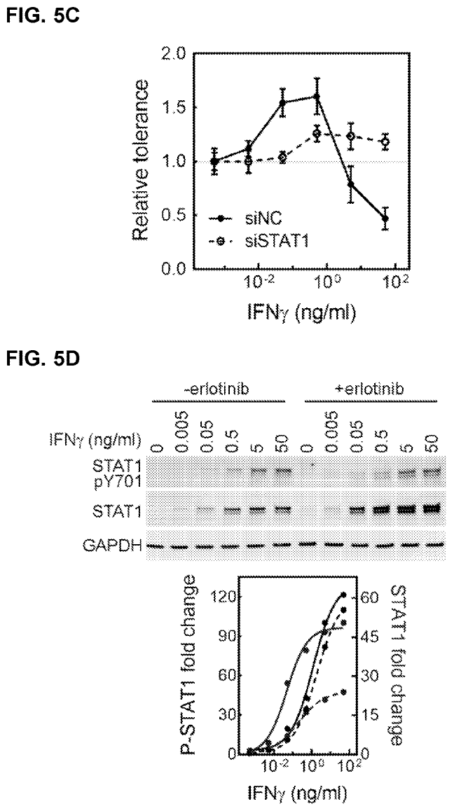

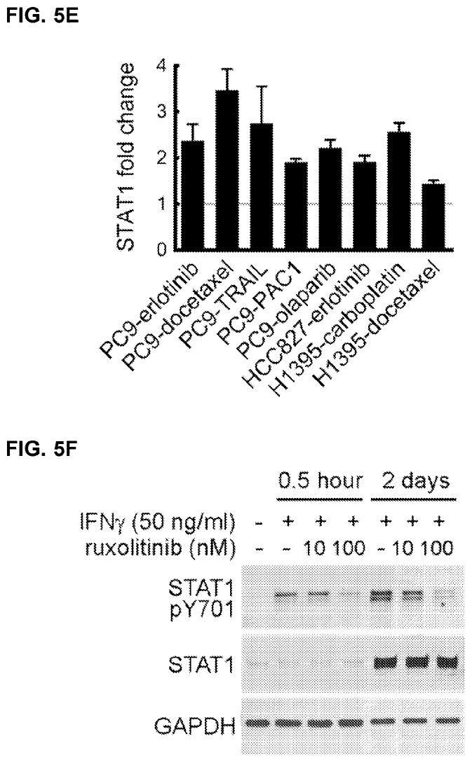

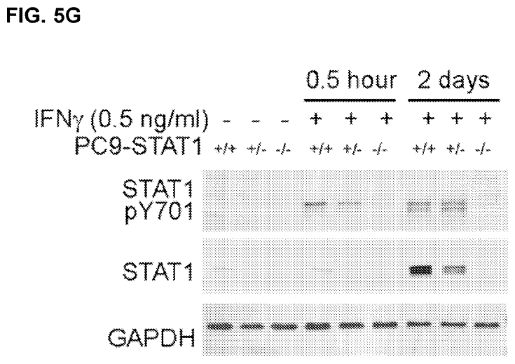

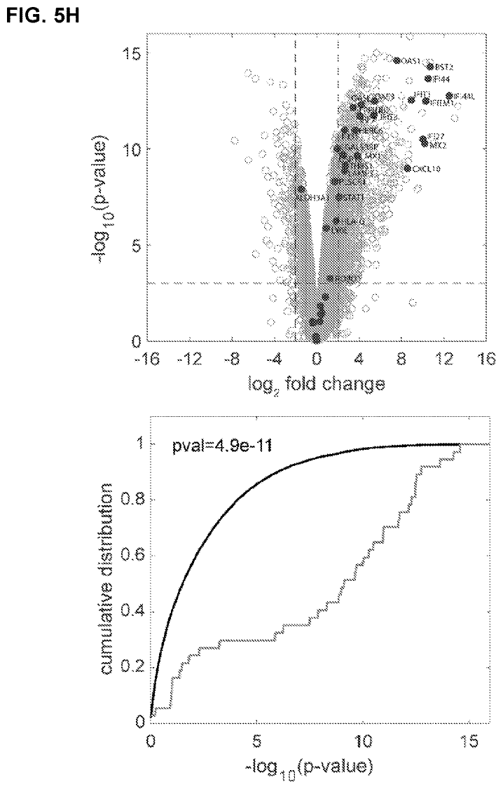

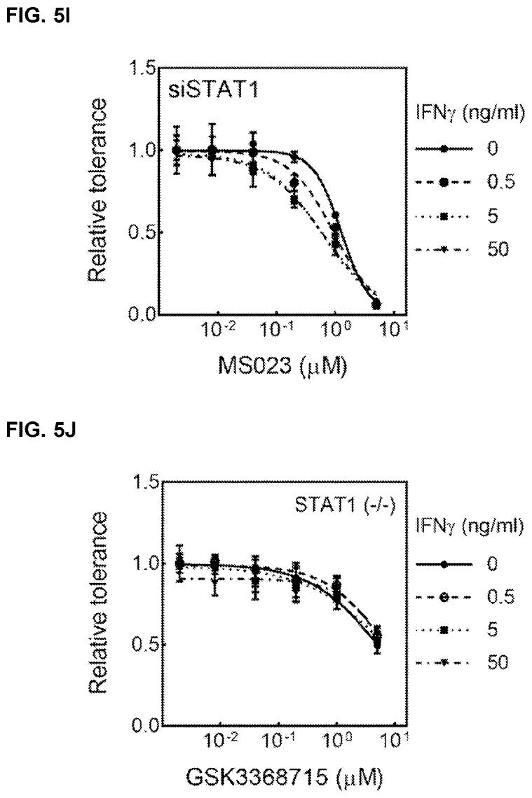

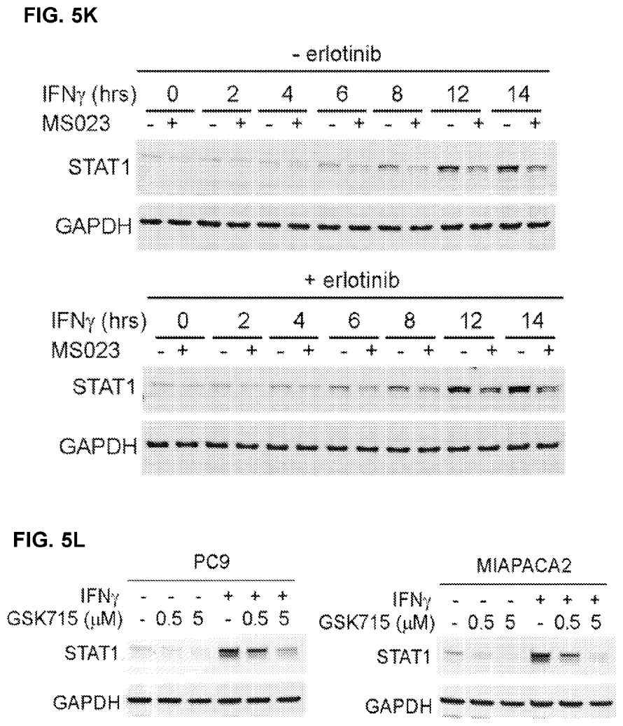

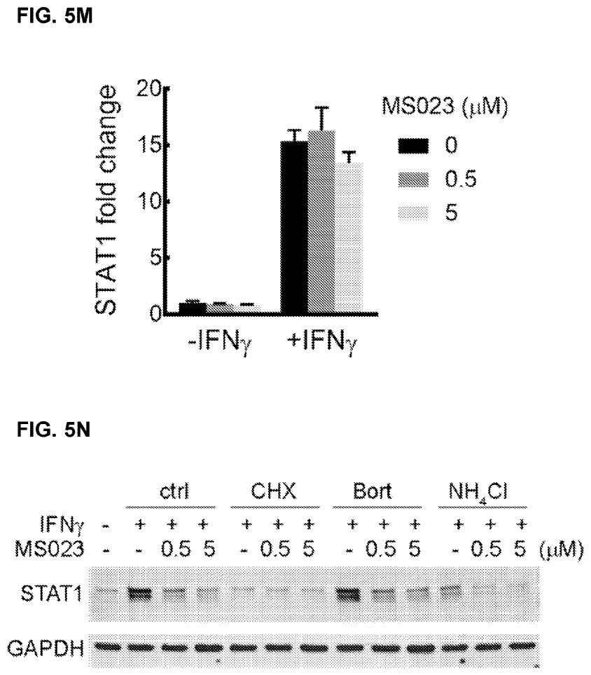

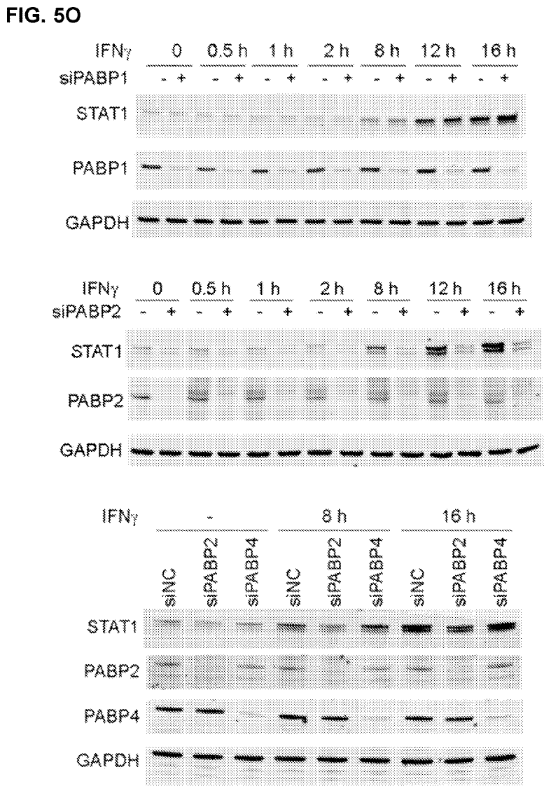

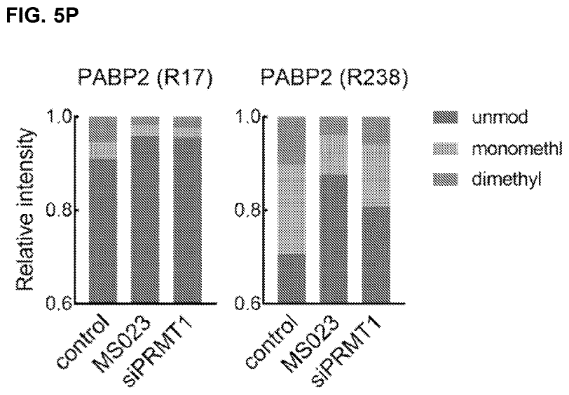

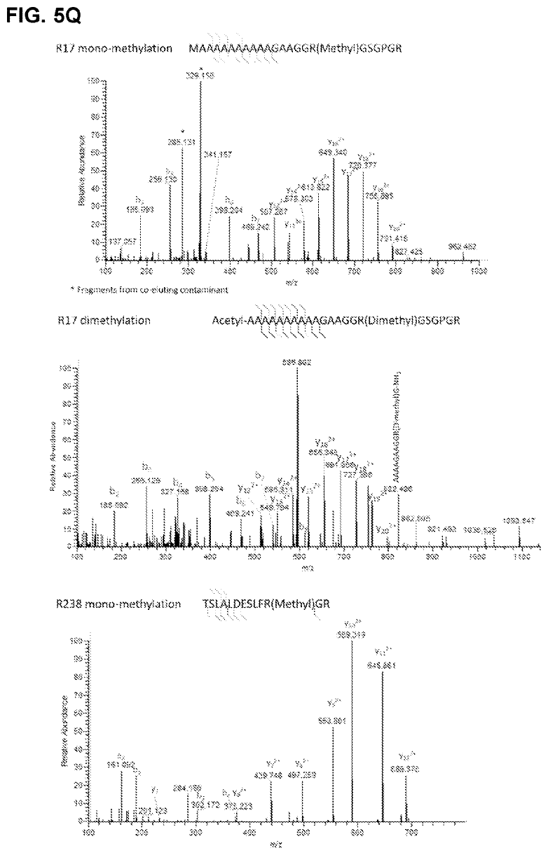

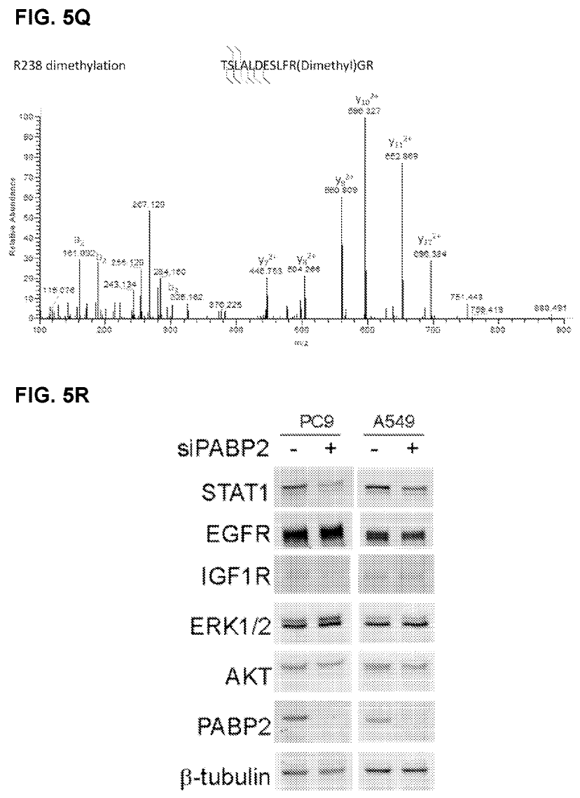

What mechanism transduces IFNγ-stimulated drug tolerance? In principle, EGFR drug tolerance could be due to bypass signaling. However, this is unlikely for IFNγ due to the generality of IFNγ's pro-survival effects across multiple cell lines and cytotoxic inducers (FIGS. 4A-4B). Further, we did not observe the reactivation of major EGFR bypass signaling (AKT, ERK and STAT3 pathway) by IFNγ (FIG. 5A). Canonical IFNγ signaling is transduced through STAT1^{18,19}. We confirmed in our PC9-erlotinib model system that IFNγ activates STAT1 signaling (i.e., rapid phosphorylation of STAT1, transcription of interferon regulatory factor 1 (IRF1), and feedback upregulation of STAT1; FIG. 5B). In fact, IFNγ signaling-modulated drug tolerance completely depends on STAT1: both pro- and anti-tolerance effects were diminished after siRNA knockdown of STAT1 (FIG. 5C); no change to drug tolerance was observed in cells with CRISPR deletion of STAT1 (FIG. 2A). Thus, IFNγ-stimulated drug tolerance depends critically on the signaling protein STAT1.

How can STAT1 transduce both pro- and anti-tolerance effects? Recent literature suggests that un-phosphorylated STAT1 (sometimes referred as U-STAT1) vs. phosphorylated P-STAT1 mediate pro- vs. anti-cancer roles^{17,20-22}. (We note that U-STAT1 level is typically measured by antibody against total STAT1 levels, which is not specific to un-phosphorylated state²³; hence, we use the terminology “total STAT1”.) We investigated whether total STAT1 promotes drug tolerance. First, erlotinib synergizes with IFNγ to increase total STAT1 as opposed to P-STAT1 (FIG. 5D). This is likely due to the induction of STAT1 expression by drug treatment-generally observed for erlotinib and other cytotoxic stressors across multiple cell lines (FIG. 5E)-consistent with reports of STAT1 as a transducer of general stress responses^{24,25}. Second, decrease of P-STAT1 by JAK inhibitor ruxolitinib (FIG. 5F) increases drug tolerance in the presence of IFNγ (FIG. 2B), suggesting an anti-tolerance function of P-STAT1. Third, heterozygous CRISPR knockout of STAT1 (FIG. 5G) decreases cell tolerance (FIG. 2C), suggesting a pro-tolerance function of STAT1 (as expected, homozygous CRISPR knockout of STAT1 shows no effect). Together, these results suggest a consistent role for total STAT1 vs. P-STAT1 mediating opposing roles of pro- vs. anti-drug tolerance.

Interestingly, cancer drug exposure has been recently reported to induce expression of interferon-stimulated genes (ISGs)²⁶. Upregulation of a subset of ISGs (the IRDS) was identified in radio-resistant cancer cells^{16,17} Analysis of publicly available transcriptomic data of drug-tolerant and -sensitive cells²⁶ revealed that most IRDS genes, including STAT1 itself, are significantly upregulated in drug-tolerant cells (Fig. S2h). This further supported our hypothesis that STAT1 could mediate cancer drug tolerance.

What is the connection between type I PRMTs and IFNγ signaling-modulated drug tolerance? The efficacy of type I PRMT inhibition by MS023 was dramatically compromised by STAT1 knockdown (compare FIG. 5I and FIG. 1E), indicating a connection between type I PRMT and STAT1. Furthermore, no efficacy of MS023 and GSK3368715 was observed in STAT1 homozygous knockout cells (compare FIGS. 2D, 5J and FIGS. 1E, 4E), suggesting the efficacy of type I PRMT inhibition depends on STAT1. Previous work in other systems identified STAT1 or its regulator PIAS1 as direct substrates of type I PRMTs²⁷, though we could not find evidence for their arginine methylation in our system (data not shown). Instead, we found inhibition of type I PRMTs by MS023 or GSK3368715 downregulated total STAT1 protein level; this was observed independent of whether IFNγ or erlotinib was present and across multiple cancer cell lines (FIG. 2E; FIGS. 5K and 5L). We found that the ability of MS023 to reduce STAT1 expression was due neither to reduction in its mRNA level (FIG. 5M) nor to autophagy- or proteasome-mediated degradation, but rather through alteration in protein synthesis (FIG. 5N). These results suggest that type I PRMTs regulate the translation of STAT1 from mRNA to protein.

By what mechanism do type I PRMTs mediate STAT1 translation? PRMTs have been previously shown to regulate RNA processing via directly methylating RNA-binding proteins (RBPs)²⁸. We found that knockdown of an RBP, poly(A)-binding protein 2 (PABP2)—but not other family members PABP1 or PABP4—dramatically reduced STAT1 expression (FIG. 2F; FIG. 5O). PABP2 was reported to be methylated on multiple arginine residues by PRMT1²⁹. The arginine methylation modulates PABP2 self-association and is suggested to mediate PABP2 function by regulating its oligomerization³⁰. Mass spectrometry experiments confirmed that PABP2 is dimethylated by type I PRMTs at multiple arginine residues, as MS023 or siRNA knockdown of PRMT1 reduced the detected dimethylation of PABP2 (FIG. 2G; FIGS. 5P and 5Q). Intriguingly, although PABP2 is reported as a general translation regulator, we observed that knockdown of PABP2 reduced the expression of STAT1, but not of other proteins we tested (FIG. 5R). Together, our results suggest methylation of PABP2 as a key mechanism by which type I PRMTs mediate STAT1 expression, which, in turn, connects type I PRMTs to STAT1-mediated drug tolerance (FIG. 2H).

### Type I PRMT Inhibition Enhances Cancer Drug Efficacy

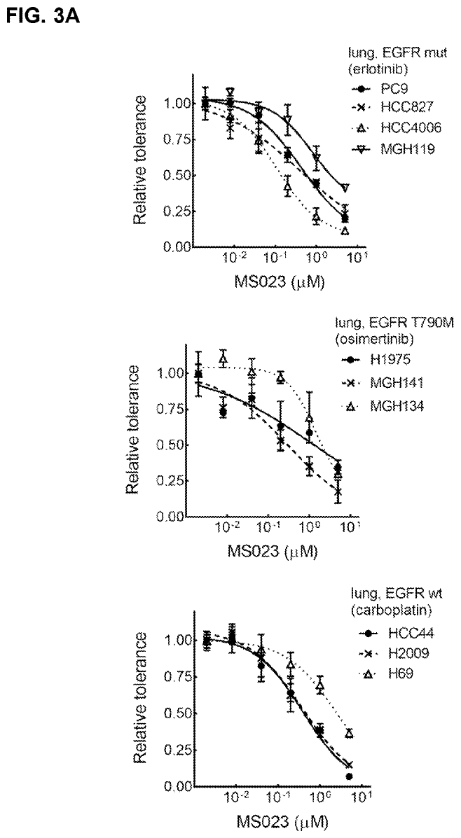

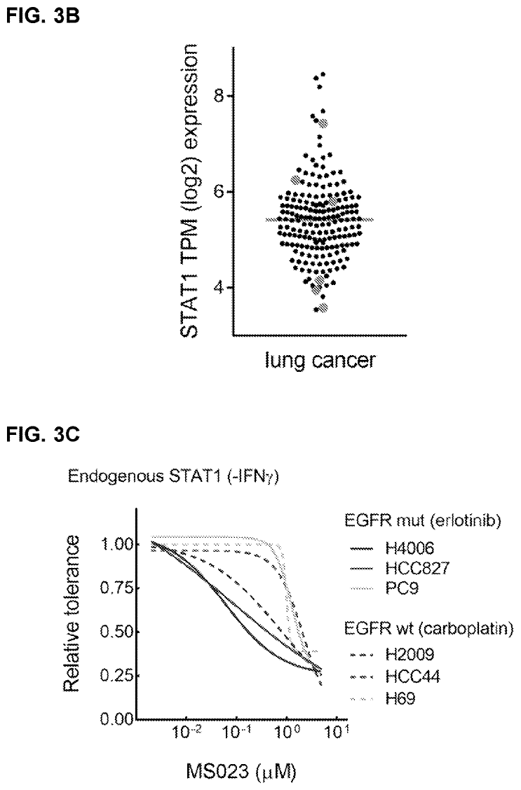

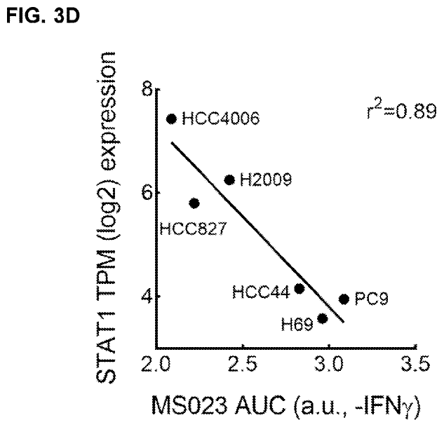

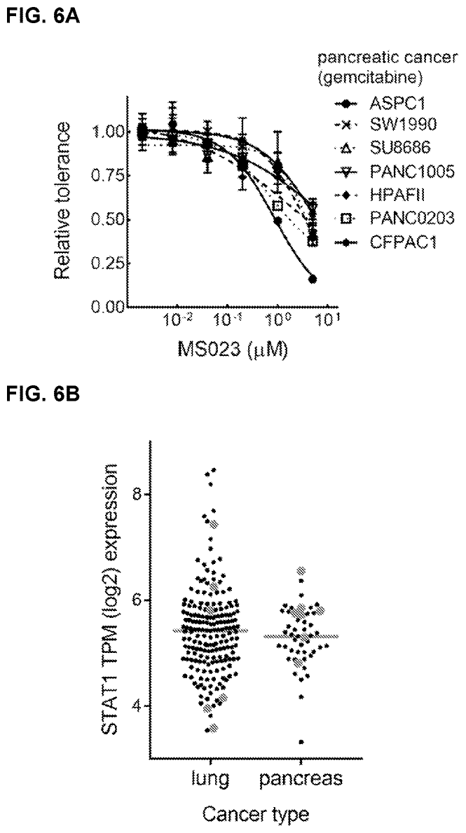

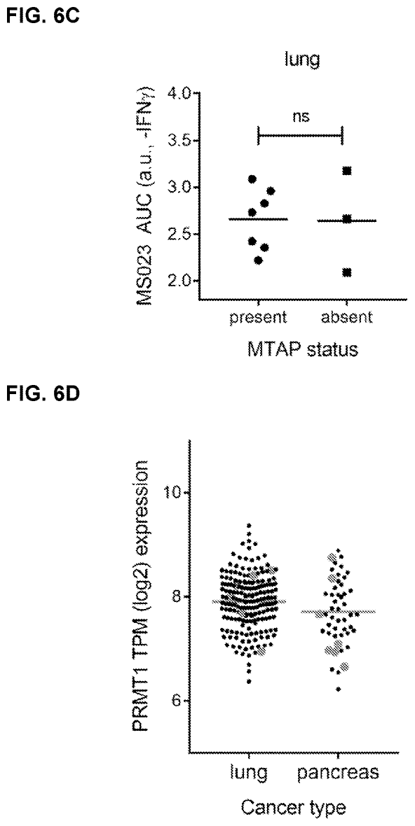

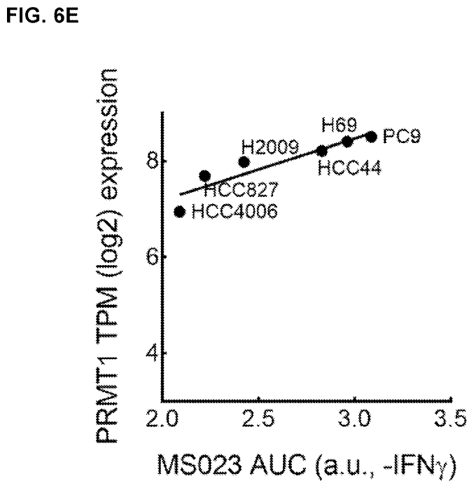

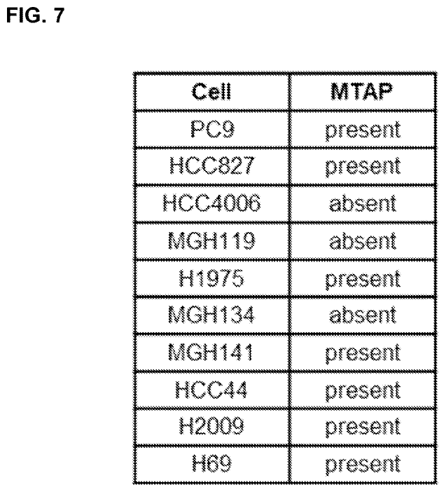

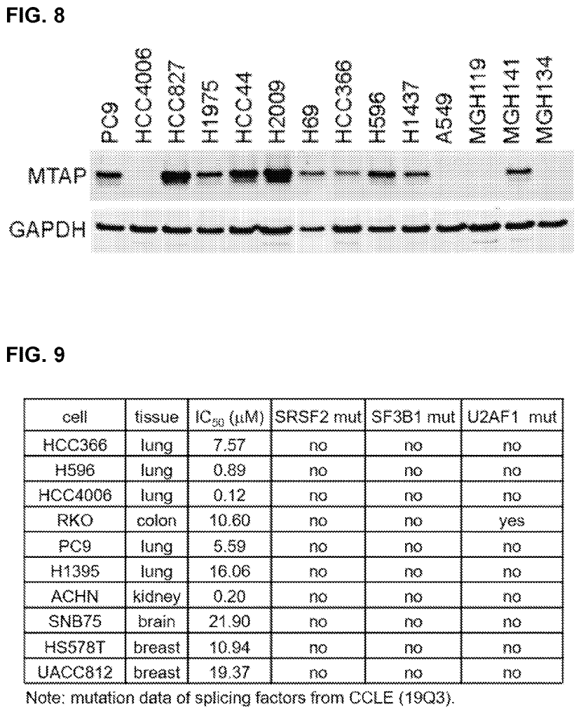

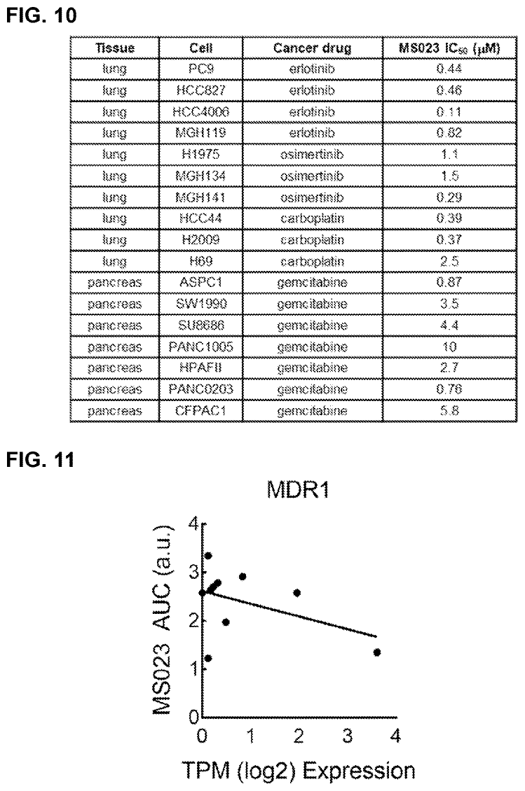

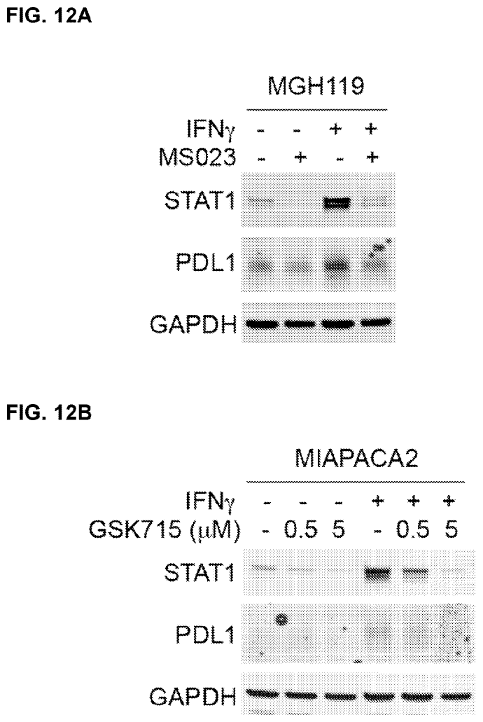

Can type I PRMT inhibition provide a general strategy for reducing drug tolerance and enhance efficacies of SOC drugs? STAT1 signaling can be activated by various sources in tumors, including interferon (IFN)-producing immune cells in the tumor microenvironment, endogenous nucleic acids, and cancer therapeutics (FIG. 2H)³¹. In conditions of IFNγ stimulation, efficacy of the type I PRMT inhibitor MS023 was observed in lung cancers for erlotinib across multiple EGFR mutant cell lines, the third-generation EGFR inhibitor osimertinib across multiple EGFR mutant cell lines with the T790M resistance mutation, and the chemotherapy drug carboplatin in EGFR wild type cell lines (FIG. 3A). We also observed efficacy of MS023 with gemcitabine in various pancreatic cancer cell lines (FIG. 6A). The IC₅₀'s of MS023 in these cell models are in the sub- to low-micromolar range, well consistent with the on-target potency of this tool compound³².

We searched for a candidate molecular indicator that suggests efficacy of type I PRMT inhibition on reducing drug tolerance. First, a natural choice is PRMT1 expression itself. However, we found that PRMT1 was ubiquitously abundant across different cell lines (compared to STAT1 expression; FIG. 6D vs. FIG. 6B) and could not explain the differential efficacies of type I PRMT inhibition (FIG. 6E). Second, loss of methylthioadenosine phosphorylase (MTAP) is a proposed biomarker for efficacy of type I PRMT inhibition as a monotherapy in some cancer types, as MTAP deficiency leads to type II PRMT inhibition and subsequent synthetic lethality with type I PRMT inhibition³³⁻³⁵. However, we found no correlation between MTAP status and the efficacy of type I PRMT inhibition on drug tolerance (FIG. 6C). Finally, we tested whether our mechanistic insights around STAT1 could provide an indicator for the efficacy of type I PRMT inhibition. We made use of a panel of lung cancer cell lines that displayed a wide range of STAT1 expression (approximately 15-fold variation; FIG. 3B) intrinsically, without experimentally added IFNγ stimulation. Excitingly, we observed a clear correlation between the anti-tolerance efficacy of type I PRMT inhibition and endogenous STAT1 expression within the cancer cells (FIGS. 3C and 3D). This suggests that STAT1 signaling may serve as a candidate in future studies focusing on biomarker discovery.

### REFERENCES

1. Sharma, S. V. et al. A chromatin-mediated reversible drug-tolerant state in cancer cell subpopulations. Cell 141, 69-80 (2010).
2. Ramirez, M. et al. Diverse drug-resistance mechanisms can emerge from drug-tolerant cancer persister cells. Nat Comms 7, 10690 (2016).
3. Boumahdi, S. & de Sauvage, F. J. The great escape: tumour cell plasticity in resistance to targeted therapy. Nat Rev Drug Discov 383, 558 (2019).
4. Luskin, M. R., Murakami, M. A., Manalis, S. R. & Weinstock, D. M. Targeting minimal residual disease: a path to cure? Nat. Rev. Cancer 18, 255-263 (2018).
5. Bivona, T. G. & Doebele, R. C. A framework for understanding and targeting residual disease in oncogene-driven solid cancers. Nat. Med. 22, 472-478 (2016).
6. Hata, A. N. et al. Tumor cells can follow distinct evolutionary paths to become resistant to epidermal growth factor receptor inhibition. Nat. Med. 22, 262-269 (2016).
7. Shaffer, S. M. et al. Rare cell variability and drug-induced reprogramming as a mode of cancer drug resistance. Nature 546, 431-435 (2017).
8. Roesch, A. et al. Overcoming Intrinsic Multidrug Resistance in Melanoma by Blocking the Mitochondrial Respiratory Chain of Slow-Cycling JARID1Bhigh Cells. Cancer Cell 23, 811-825 (2013).
9. Paek, A. L., Liu, J. C., Loewer, A., Forrester, W. C. & Lahav, G. Cell-to-Cell Variation in p53 Dynamics Leads to Fractional Killing. Cell 165, 631-642 (2016).
10. Spencer, S. L., Gaudet, S., Albeck, J. G., Burke, J. M. & Sorger, P. K. Non-genetic origins of cell-to-cell variability in TRAIL-induced apoptosis. Nature 459, 428-432 (2009).
11. Russo, M. et al. Adaptive mutability of colorectal cancers in response to targeted therapies. Science 40, eaav4474 (2019).
12. Roesch, A. et al. A temporarily distinct subpopulation of slow-cycling melanoma cells is required for continuous tumor growth. Cell 141, 583-594 (2010).
13. Kurata, T. et al. Effect of re-treatment with gefitinib (‘Iressa’, ZD1839) after acquisition of resistance. Ann. Oncol. 15, 173-174 (2004).
14. Yano, S. et al. Retreatment of lung adenocarcinoma patients with gefitinib who had experienced favorable results from their initial treatment with this selective epidermal growth factor receptor inhibitor: a report of three cases. Oncol. Res. 15, 107-111 (2005).
15. Korc, M. & Friesel, R. E. The role of fibroblast growth factors in tumor growth. Curr Cancer Drug Targets 9, 639-651 (2009).
16. Weichselbaum, R. R. et al. An interferon-related gene signature for DNA damage resistance is a predictive marker for chemotherapy and radiation for breast cancer. Proc. Natl. Acad. Sci. U.S.A. 105, 18490-18495 (2008).
17. Khodarev, N. N. et al. STAT1 is overexpressed in tumors selected for radioresistance and confers protection from radiation in transduced sensitive cells. Proc. Natl. Acad. Sci. USA. 101, 1714-1719 (2004).
18. Platanias, L. C. Mechanisms of type-I- and type-II-interferon-mediated signalling. Nat. Rev. Immunol. 5, 375-386 (2005).
19. Schroder, K., Hertzog, P. J., Ravasi, T. & Hume, D. A. Interferon-gamma: an overview of signals, mechanisms and functions. J. Leukoc. Biol. 75, 163-189 (2004).
20. Khodarev, N. N., Roizman, B. & Weichselbaum, R. R. Molecular pathways: interferon/stat1 pathway: role in the tumor resistance to genotoxic stress and aggressive growth. Clinical Cancer Research 18, 3015-3021 (2012).
21. Zimmerman, M. A. et al. Unphosphorylated STAT1 promotes sarcoma development through repressing expression of Fas and bad and conferring apoptotic resistance. Cancer Res. 72, 4724-4732 (2012).
22. Cheon, H., Yang, J. & Stark, G. R. The functions of signal transducers and activators of transcriptions 1 and 3 as cytokine-inducible proteins. J. Interferon Cytokine Res. 31, 33-40 (2011).
23. Cheon, H. & Stark, G. R. Unphosphorylated STAT1 prolongs the expression of interferon-induced immune regulatory genes. Proc. Natl. Acad. Sci. U.S.A. 106, 9373-9378 (2009).
24. Kovarik, P. et al. Stress-induced phosphorylation of STAT1 at Ser727 requires p38 mitogen-activated protein kinase whereas IFN-gamma uses a different signaling pathway. Proceedings of the National Academy of Sciences 96, 13956-13961 (1999).
25. Dudley, A. C., Thomas, D., Best, J. & Jenkins, A. The STATs in cell stress-type responses. Cell Commun. Signal 2, 8-5 (2004).
26. Guler, G. D. et al. Repression of Stress-Induced LINE-1 Expression Protects Cancer Cell Subpopulations from Lethal Drug Exposure. Cancer Cell 32, 221-237.e13 (2017).
27. Mowen, K. A. et al. Arginine methylation of STAT1 modulates IFNalpha/beta-induced transcription. Cell 104, 731-741 (2001).
28. Bedford, M. T. & Clarke, S. G. Protein arginine methylation in mammals: who, what, and why. Mol. Cell 33, 1-13 (2009).
29. Smith, J. J. et al. Unusual sites of arginine methylation in Poly(A)-binding protein II and in vitro methylation by protein arginine methyltransferases PRMT1 and PRMT3. J. Biol. Chem. 274, 13229-13234 (1999).
30. Perreault, A., Lemieux, C. & Bachand, F. Regulation of the nuclear poly(A)-binding protein by arginine methylation in fission yeast. J. Biol. Chem. 282, 7552-7562 (2007).
31. Cheon, H., Borden, E. C. & Stark, G. R. Interferons and their stimulated genes in the tumor microenvironment. Semin. Oncol. 41, 156-173 (2014).
32. Eram, M. S. et al. A Potent, Selective, and Cell-Active Inhibitor of Human Type I Protein Arginine Methyltransferases. ACS Chem. Biol. 11, 772-781 (2016).
33. Fedoriw, A. et al. Anti-tumor Activity of the Type I PRMT Inhibitor, GSK3368715, Synergizes with PRMT5 Inhibition through MTAP Loss. Cancer Cell 36, 100-114.e25 (2019).
34. Kryukov, G. V. et al. MTAP deletion confers enhanced dependency on the PRMT5 arginine methyltransferase in cancer cells. Science 351, 1214-1218 (2016).
35. Marjon, K. et al. MTAP Deletions in Cancer Create Vulnerability to Targeting of the MAT2A/PRMT5/RIOK1 Axis. Cell Rep 15, 574-587 (2016).

### P EMBODIMENTS

P Embodiment 1. A method of treating cancer in a subject having elevated STAT1 activity, said method comprising administering a therapeutically effective amount of a type I PRMT inhibitor to said subject.
P Embodiment 2. The method of P embodiment 1, wherein said subject is or has been treated with an anti-cancer agent.
P Embodiment 3. The method of P embodiments 1 or 2, wherein said subject has elevated IRF1 activity, elevated SOCS1 activity, elevated APOL1 activity, elevated B2M activity, elevated GBP activity, and/or elevated RNF213 activity.
P Embodiment 4. The method of any one of P embodiments 1-3, wherein said subject has elevated interferon gamma (IFNγ) levels.
P Embodiment 5. The method of any one of P embodiments 1-4, wherein said subject has a STAT1 activating genetic mutation.
P Embodiment 6. The method of P embodiment 5, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.
P Embodiment 7. The method of any one of P embodiment 1-6, wherein said subject comprises a tumor, said tumor comprising CD4+ T cells, CD8+ T cells, macrophages, neutrophils, and/or NK cells.
P Embodiment 8. The method of any one of P embodiments 1-7, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.
P Embodiment 9. The method of any one of P embodiments 1-8, wherein said subject comprises a tumor, said tumor comprising TAP, B2M, CIITA, HLA-DR, and/or HLA-DMA.
P Embodiment 10. A method of treating cancer in a subject, said method comprising administering a therapeutically effective amount of a type I PRMT inhibitor to said subject, wherein said subject has been previously treated with a STAT1 activating compound.
P Embodiment 11. A method of treating cancer in a subject, said method comprising administering a therapeutically effective amount of a type I PRMT inhibitor and a STAT1 activating compound to said subject.
P Embodiment 12. The method of P embodiment 10 or 11, wherein said STAT1 activating compound is an anti-cancer agent.
P Embodiment 13. A method of treating cancer in a subject in need thereof, said method comprising: (i) detecting an elevated STAT1 activity in a subject; and (ii) administering a therapeutically effective amount of a type I PRMT inhibitor to said subject.
P Embodiment 14. A method of treating cancer in a subject in need thereof, said method comprising: (i) detecting an elevated STAT1 activity in a subject; and (ii) administering a therapeutically effective amount of an anti-cancer agent to said subject.
P Embodiment 15. A method of treating cancer in a subject in need thereof, said method comprising: (i) detecting a STAT1 activity in a subject; and (ii) administering a therapeutically effective amount of a STAT1 activating compound to said subject.
P Embodiment 16. The method of P embodiment 15, further comprising administering to said subject an effective amount of a type I PRMT inhibitor.
P Embodiment 17. The method of P embodiment 15 or 16, wherein said STAT1 activating compound is an anti-cancer agent.
P Embodiment 18. The method of any one of P embodiments 13-16, wherein said subject is or has been treated with an anti-cancer agent.
P Embodiment 19. The method of any one of P embodiments 13-18, wherein said subject has elevated IRF1 activity, elevated SOCS1 activity, elevated APOL1 activity, elevated B2M activity, elevated GBP activity, and/or elevated RNF213 activity.
P Embodiment 20. The method of any one of P embodiments 13-19, wherein said subject has elevated interferon gamma (IFNγ) levels.
P Embodiment 21. The method of any one of P embodiments 13-20, wherein said subject has a STAT1 activating genetic mutation.
P Embodiment 22. The method of P embodiment 21, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.
P Embodiment 23. The method of any one of P embodiments 13-22, wherein said subject comprises a tumor, said tumor comprising CD4+ T cells, CD8+ T cells, macrophages, neutrophils, and/or NK cells.
P Embodiment 24. The method of any one of P embodiments 13-23, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.
P Embodiment 25. The method of any one of P embodiments 13-24, wherein said subject comprises a tumor, said tumor comprising TAP, B2M, CIITA, HLA-DR, and/or HLA-DMA.

### EMBODIMENTS

Embodiment 1. A method of treating cancer in a subject having elevated STAT1 activity, said method comprising administering a therapeutically effective amount of a type I PRMT inhibitor to said subject.
Embodiment 2. The method of embodiment 1, wherein said subject is or has been treated with an anti-cancer agent.
Embodiment 3. The method of embodiment 2, wherein said anti-cancer agent is erlotinib, osimertinib, carboplatin, or gemcitabine.
Embodiment 4. The method of any one of embodiments 1-3, wherein said type I PRMT inhibitor has the structure of formula:

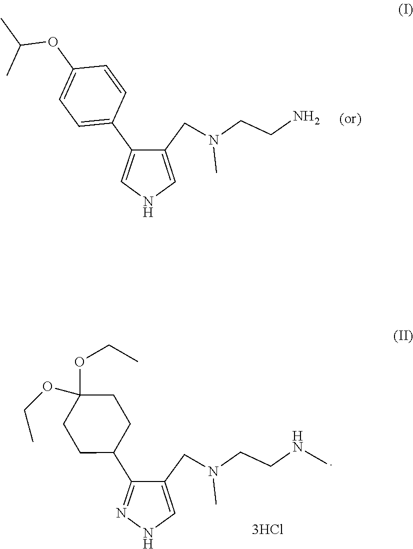 [SMILES: CC(C)Oc1ccc(-c2c[nH]cc2CN(C)CCN)cc1 ; CCOC1(OCC)CCC(c2n[nH]cc2CN(C)CCNC)CC1]

Embodiment 5. The method of any one of embodiments 1-4, wherein said cancer is lung cancer, colon cancer, kidney cancer, brain cancer, breast cancer, or pancreatic cancer.
Embodiment 6. The method of any one of embodiments 1-5, wherein said subject has elevated IRF1 activity, elevated SOCS1 activity, elevated APOL1 activity, elevated B2M activity, elevated GBP activity, and/or elevated RNF213 activity.
Embodiment 7. The method of any one of embodiments 1-5, wherein said subject has elevated interferon alpha (IFNα) levels, interferon gamma (IFN [symbol] ) levels, epidermal growth factor (EGF) levels, platelet derived growth factor (PDGF) levels, or interleukin 6 (IL6) levels.
Embodiment 8. The method of any one of embodiments 1-7, wherein said subject has a STAT1 activating genetic mutation.
Embodiment 9. The method of embodiment 8, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.
Embodiment 10. The method of any one of embodiments 1-9, wherein said subject comprises a tumor, said tumor comprising CD4+ T cells, CD8+ T cells, macrophages, neutrophils, and/or NK cells.
Embodiment 11. The method of any one of embodiments 1-10, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.
Embodiment 12. The method of any one of embodiments 1-11, wherein said subject comprises a tumor, said tumor comprising TAP, B2M, CIITA, HLA-DR, and/or HLA-DMA.
Embodiment 13. A method of treating cancer in a subject, said method comprising administering a therapeutically effective amount of a type I PRMT inhibitor to said subject, wherein said subject has been previously treated with a STAT1 activating compound.
Embodiment 14. A method of treating cancer in a subject, said method comprising administering a therapeutically effective amount of a type I PRMT inhibitor and a STAT1 activating compound to said subject.
Embodiment 15. The method of embodiment 13 or 14, wherein said STAT1 activating compound is an anti-cancer agent.
Embodiment 16. The method of embodiment 15, wherein said anti-cancer agent is erlotinib, osimertinib, carboplatin, or gemcitabine.
Embodiment 17. The method of embodiment 13 or 14, wherein said type I PRMT inhibitor has the structure of formula:

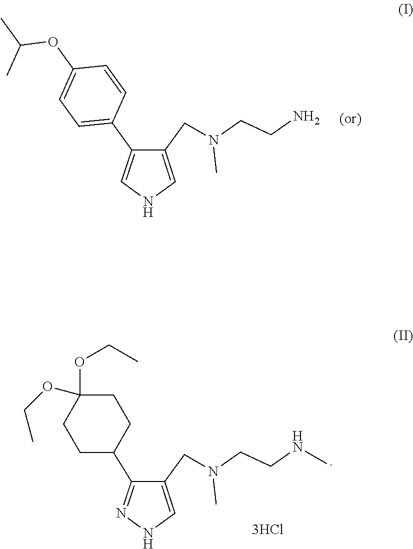 [SMILES: CC(C)Oc1ccc(-c2c[nH]cc2CN(C)CCN)cc1 ; CCOC1(OCC)CCC(c2n[nH]cc2CN(C)CCNC)CC1]

Embodiment 18. The method of embodiment 13 or 14, wherein said cancer is lung cancer, colon cancer, kidney cancer, brain cancer, breast cancer, or pancreatic cancer.
Embodiment 19. A method of treating cancer in a subject in need thereof, said method comprising: (i) detecting an elevated STAT1 activity in a subject; and (ii) administering a therapeutically effective amount of a type I PRMT inhibitor to said subject.
Embodiment 20. A method of treating cancer in a subject in need thereof, said method comprising: (i) detecting an elevated STAT1 activity in a subject; and (ii) administering a therapeutically effective amount of an anti-cancer agent to said subject.
Embodiment 21. A method of treating cancer in a subject in need thereof, said method comprising: (i) detecting a STAT1 activity in a subject; and (ii) administering a therapeutically effective amount of a STAT1 activating compound to said subject.
Embodiment 22. The method of embodiment 21, further comprising administering to said subject an effective amount of a type I PRMT inhibitor.
Embodiment 23. The method of embodiment 19 or 22, wherein said type I PRMT inhibitor has the structure of formula:

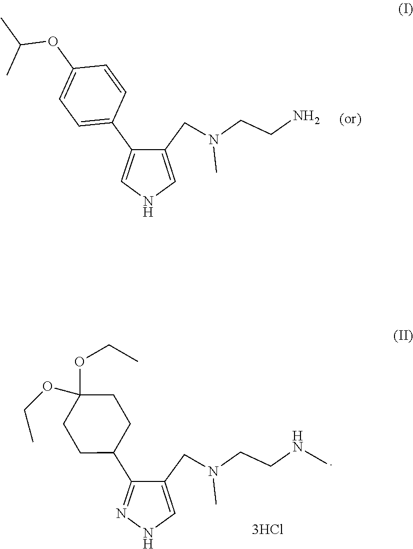 [SMILES: CC(C)Oc1ccc(-c2c[nH]cc2CN(C)CCN)cc1 ; CCOC1(OCC)CCC(c2n[nH]cc2CN(C)CCNC)CC1]

Embodiment 24. The method of embodiment 21 or 22, wherein said STAT1 activating compound is an anti-cancer agent.
Embodiment 25. The method of embodiment 24, wherein said anti-cancer agent is erlotinib, osimertinib, carboplatin, or gemcitabine.
Embodiment 26. The method of any one of embodiments 19-22, wherein said subject is or has been treated with an anti-cancer agent.
Embodiment 27. The method of embodiment 26, wherein said anti-cancer agent is erlotinib, osimertinib, carboplatin, or gemcitabine.
Embodiment 28. The method of any one of embodiments 19-22, wherein said cancer is lung cancer, colon cancer, kidney cancer, brain cancer, breast cancer, or pancreatic cancer.
Embodiment 29. The method of any one of embodiments 19-28, wherein said subject has elevated IRF1 activity, elevated SOCS1 activity, elevated APOL1 activity, elevated B2M activity, elevated GBP activity, and/or elevated RNF213 activity.
Embodiment 30. The method of any one of embodiments 19-28, wherein said subject has elevated interferon alpha (IFNα) levels, interferon gamma (IFN [symbol] ) levels, epidermal growth factor (EGF) levels, platelet derived growth factor (PDGF) levels, or interleukin 6 (IL6) levels.
Embodiment 31 The method of any one of embodiments 19-30, wherein said subject has a STAT1 activating genetic mutation.
Embodiment 32. The method of embodiment 31, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.
Embodiment 33. The method of any one of embodiments 19-32, wherein said subject comprises a tumor, said tumor comprising CD4+ T cells, CD8+ T cells, macrophages, neutrophils, and/or NK cells.
Embodiment 34. The method of any one of embodiments 19-33, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.
Embodiment 35. The method of any one of embodiments 19-34, wherein said subject comprises a tumor, said tumor comprising TAP, B2M, CIITA, HLA-DR, and/or HLA-DMA.

What is claimed is:

1. A method of treating cancer in a subject having elevated STAT1 activity, said method comprising administering a therapeutically effective amount of a type I PRMT inhibitor to said subject.

2. The method of claim 1, wherein said subject is or has been treated with an anti-cancer agent.

3. The method of claim 2, wherein said anti-cancer agent is erlotinib, osimertinib, carboplatin, or gemcitabine.

4. The method of any one of claims 1-3, wherein said type I PRMT inhibitor has the structure of formula:

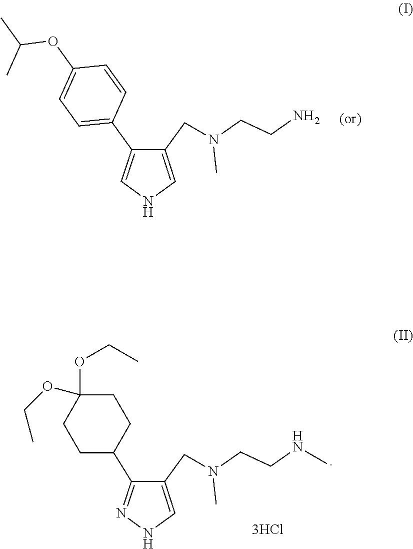 [SMILES: CC(C)Oc1ccc(-c2c[nH]cc2CN(C)CCN)cc1 ; CCOC1(OCC)CCC(c2n[nH]cc2CN(C)CCNC)CC1]

5. The method of any one of claims 1-4, wherein said cancer is lung cancer, colon cancer, kidney cancer, brain cancer, breast cancer, or pancreatic cancer.

6. The method of any one of claims 1-5, wherein said subject has elevated IRF1 activity, elevated SOCS1 activity, elevated APOL1 activity, elevated B2M activity, elevated GBP activity, and/or elevated RNF213 activity.

7. The method of any one of claims 1-5, wherein said subject has elevated interferon alpha (IFNα) levels, interferon gamma (IFNγ) levels, epidermal growth factor (EGF) levels, platelet derived growth factor (PDGF) levels, or interleukin 6 (IL6) levels.

8. The method of any one of claims 1-7, wherein said subject has a STAT1 activating genetic mutation.

9. The method of claim 8, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.

10. The method of any one of claims 1-9, wherein said subject comprises a tumor, said tumor comprising CD4+ T cells, CD8+ T cells, macrophages, neutrophils, and/or NK cells.

11. The method of any one of claims 1-10, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.

12. The method of any one of claims 1-11, wherein said subject comprises a tumor, said tumor comprising TAP, B2M, CIITA, HLA-DR, and/or HLA-DMA.

13. A method of treating cancer in a subject, said method comprising administering a therapeutically effective amount of a type I PRMT inhibitor to said subject, wherein said subject has been previously treated with a STAT1 activating compound.

14. A method of treating cancer in a subject, said method comprising administering a therapeutically effective amount of a type I PRMT inhibitor and a STAT1 activating compound to said subject.

15. The method of claim 13 or 14, wherein said STAT1 activating compound is an anti-cancer agent.

16. The method of claim 15, wherein said anti-cancer agent is erlotinib, osimertinib, carboplatin, or gemcitabine.

17. The method of claim 13 or 14, wherein said type I PRMT inhibitor has the structure of formula:

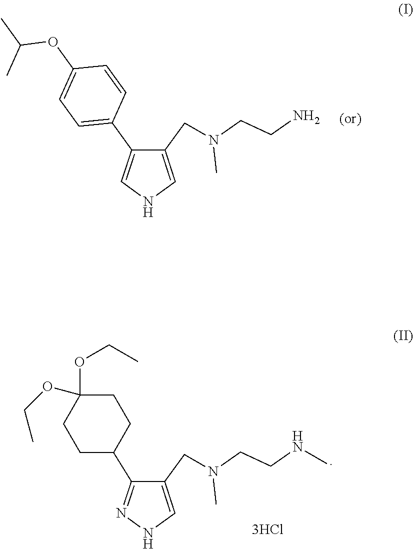 [SMILES: CC(C)Oc1ccc(-c2c[nH]cc2CN(C)CCN)cc1 ; CCOC1(OCC)CCC(c2n[nH]cc2CN(C)CCNC)CC1]

18. The method of claim 13 or 14, wherein said cancer is lung cancer, colon cancer, kidney cancer, brain cancer, breast cancer, or pancreatic cancer.

19. A method of treating cancer in a subject in need thereof, said method comprising:
(i) detecting an elevated STAT1 activity in a subject; and
(ii) administering a therapeutically effective amount of a type I PRMT inhibitor to said subject.

20. A method of treating cancer in a subject in need thereof, said method comprising:
(i) detecting an elevated STAT1 activity in a subject; and
(ii) administering a therapeutically effective amount of an anti-cancer agent to said subject.

21. A method of treating cancer in a subject in need thereof, said method comprising:
(i) detecting a STAT1 activity in a subject; and
(ii) administering a therapeutically effective amount of a STAT1 activating compound to said subject.

22. The method of claim 21, further comprising administering to said subject an effective amount of a type I PRMT inhibitor.

23. The method of claim 19 or 22, wherein said type I PRMT inhibitor has the structure of formula:

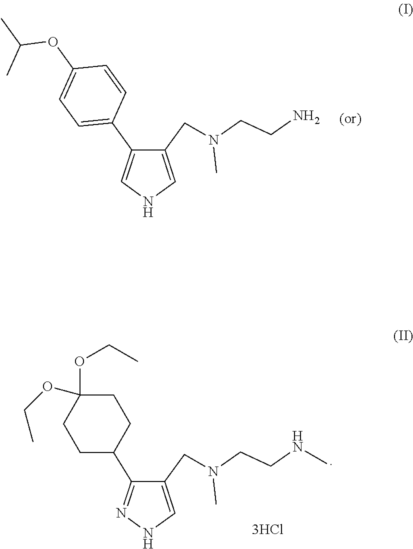 [SMILES: CC(C)Oc1ccc(-c2c[nH]cc2CN(C)CCN)cc1 ; CCOC1(OCC)CCC(c2n[nH]cc2CN(C)CCNC)CC1]

24. The method of claim 21 or 22, wherein said STAT1 activating compound is an anti-cancer agent.

25. The method of claim 24, wherein said anti-cancer agent is erlotinib, osimertinib, carboplatin, or gemcitabine.

26. The method of any one of claims 19-22, wherein said subject is or has been treated with an anti-cancer agent.

27. The method of claim 26, wherein said anti-cancer agent is erlotinib, osimertinib, carboplatin, or gemcitabine.

28. The method of any one of claims 19-22, wherein said cancer is lung cancer, colon cancer, kidney cancer, brain cancer, breast cancer, or pancreatic cancer.

29. The method of any one of claims 19-28, wherein said subject has elevated IRF1 activity, elevated SOCS1 activity, elevated APOL1 activity, elevated B2M activity, elevated GBP activity, and/or elevated RNF213 activity.

30. The method of any one of claims 19-28, wherein said subject has elevated interferon alpha (IFNα) levels, interferon gamma (IFNγ) levels, epidermal growth factor (EGF) levels, platelet derived growth factor (PDGF) levels, or interleukin 6 (IL6) levels.

31. The method of any one of claims 19-30, wherein said subject has a STAT1 activating genetic mutation.

32. The method of claim 31, wherein said STAT1 activating genetic mutation is a JAK1/2 mutation, an IFNGR1/2 mutation, a B2M mutation, a 9p21.33 disruption, a CDKN2A deletion, a 5q deletion, an IRF1 inactivating rearrangement, an IRF1 deletion, a SOCS1 mutation or a SOCS1 amplification.

33. The method of any one of claims 19-32, wherein said subject comprises a tumor, said tumor comprising CD4+ T cells, CD8+ T cells, macrophages, neutrophils, and/or NK cells.

34. The method of any one of claims 19-33, wherein said subject comprises a tumor, said tumor comprising PD-1, PD-L1/2, CD155, CD80/86, CD28, CTLA-4, galectin-9, TIM3, Siglec-15, ICOS, ICOS-L, CD47, CD70, and/or 4-1BBL.

35. The method of any one of claims 19-34, wherein said subject comprises a tumor, said tumor comprising TAP, B2M, CIITA, HLA-DR, and/or HLA-DMA.
